(19) United States
(12) Patent Application Publication
(10) Pub. No.: US 2025/0032723 A1
(43) Pub. Date: Jan. 30, 2025

(54) DRUG DELIVERY DEVICE

(71) Applicant: Sanofi, Paris (FR)

(72) Inventors: Stefan Alt, Frankfurt am Main (DE); Tim Glässer, Rüsselsheim (DE); Michael Helmer, Frankfurt am Main (DE); Stephan Mücke, Rüsselsheim (DE); Peter Nober, Rüsselsheim (DE); Michael Schabbach, Frankfurt am Main (DE); Martin Vitt, Frankfurt am Main (DE)

(21) Appl. No.: 18/706,862

(22) PCT Filed: Nov. 1, 2022
(86) PCT No.: PCT/EP2022/080401

§ 371 Date: May 2, 2024

(30) Foreign Application Priority Data
Nov. 3, 2021 (EP) 21315214.3

(51) Int. Cl. A61M 5/315

(52) U.S. Cl. CPC A61M 5/31541; A61M 5/31551; A61M 5/3157; A61M 5/3158

(57) ABSTRACT

A drug delivery device includes a mechanism unit with a housing element, a first movable element that is movable with respect to the housing element, and an electromechanical actuator that, when operated, moves an actuator element between a first position and a second position. The mechanism unit is configured to: operatively couple with a drug reservoir unit, enable a dispense process for dispensing a drug dose, and enable setting a drug dose to be dispensed. Setting a drug dose involves movement of the first movable element in a first direction. The mechanism unit is configured to block movement of the first movable element in the first direction when the actuator element is in the first position to prevent setting a drug dose and to allow movement of the first movable element in the first direction when the actuator element is in the second position.

### CROSS REFERENCE TO RELATED APPLICATIONS

The present application is the national stage entry of International Patent Application No. PCT/EP2022/080401, filed on Nov. 1, 2022, and claims priority to Application No. EP 21315214.3, filed on Nov. 3, 2021, the disclosures of which are incorporated herein by reference.

### TECHNICAL FIELD

The present disclosure relates to a drug delivery device.

### BACKGROUND

Administering an injection is a process which presents a number of risks and challenges for users and healthcare professionals, both mental and physical. A drug delivery device can make self-injection easier for patients. Safe operation of the drug delivery device is desirable.

### SUMMARY

The present disclosure relates to an improved drug delivery device, such as a drug delivery device which provides a safe operation for the user.

The drug delivery device described herein may be an injection device. The drug delivery device may be an autoinjector and/or a variable dose device or a fixed dose device and/or a pen type device, e.g., a dial extension pen.

According to some embodiments, the drug delivery device includes a mechanism unit. The mechanism unit may include a dispense mechanism for dispensing a drug dose and/or a setting mechanism for setting a drug dose.

The dispense mechanism and/or the setting mechanism may include several elements which interact with each other during dose dispensing or dose setting. For example, a coupling between two or more elements of the mechanism unit is changed when switching from dose setting to dose dispensing or vice versa. Two or more elements are splined during dose setting such that they are rotationally fixed to each other, where the splined coupling is released for dose dispensing so that these elements rotate relative to each other during dose dispensing.

The dispense mechanism may include a plunger rod configured to act on a drug reservoir to dispense a drug dose. The mechanism unit may be configured such that the plunger rod moves axially in a distal direction during dose dispensing. The plunger rod may also rotate during dose dispensing, e.g., due to a threaded engagement with a further element of the mechanism unit, like a drive element. During setting a drug dose, the plunger rod does not move.

The dispense mechanism may also include an energy member to provide energy for dispensing a drug dose. The energy member may provide energy for moving the plunger rod in a distal direction. In some examples, the energy member is a drive spring, like a compression spring or a torsion spring, or a gas cartridge or an electric motor. Alternatively, no additional energy member for moving the plunger rod is used. The force to move the plunger rod and dispensing a drug dose may thus be provided by the user.

The dispense mechanism may include a drive element, e.g., a drive sleeve. The drive sleeve may circumferentially surround the plunger rod. The drive element may be threadedly engaged with the plunger rod. During dispensing a drug dose, the drive element may move in distal direction, e.g., without rotation, and may thereby force the plunger rod to rotate and also move in a distal direction.

The setting mechanism may include a setting element, e.g., a dial sleeve and/or a number sleeve. During dose setting, the drive element may be splined to the setting element. For example, during dose setting, the drive element and the setting element move together, e.g., on a helical path, in a proximal direction but may not move relative to each other. During dose dispensing, the splined coupling between the drive element and the setting element may be released. During dose dispensing, the setting element moves back on the helical path in distal direction, but the drive element only moves axially in distal direction without rotation. For realizing the splined coupling and for releasing the splined coupling between the drive element and the setting element, the mechanism unit may include a clutch and/or a clicker arrangement and/or a clutch spring.

The mechanism unit may include a user interface member configured to be operated by a user, e.g., to be touched by a user, to dispense a drug dose. For example, the user interface member is a button or a knob. To dispense a drug dose, the user interface member is pushed in a distal direction by the user. This user interface member may also be referred to as dose dispense member.

The mechanism unit may also include a user interface member configured to be operated by a user, e.g., to be touched by a user, to set a drug dose. For setting a drug dose, the user may rotate and/or move the user interface member in a proximal direction. This user interface member may also be referred to as dose setting member.

The user interface member for setting a drug dose may be the same as the user interface member for dispensing a drug dose.

According to some embodiments, the mechanism unit includes a housing element. The housing element may be a sleeve that circumferentially surrounds some or all of the elements of the mechanism unit. The housing element may include an outer surface which forms an outer surface of the drug delivery device that can be touched by a user.

According to some embodiments, the mechanism unit includes a first movable element. The first movable element is movable with respect to the housing element. In some embodiments, the first movable element is rotatably and/or axially movable with respect to the housing element. The first movable element may be assigned to the setting mechanism and/or to the dispense mechanism. For example, the first movable element is one of: the drive element, the setting element, the plunger rod, the dose setting member, the dose dispense member.

Unless stated otherwise, movement of a member, element, or feature means a movement relative to the housing element.

According to some embodiments, the mechanism unit includes an electromechanical actuator. The actuator may be configured such that, when operated, an actuator element of the actuator is moved between a first position and a second position.

As used herein, the term electromechanical actuator refers to an actuator which converts an electrical signal into a movement of an actuator element. For example, when operated, the actuator may move the actuator element from the first position into the second position and/or vice versa. The movement between the first position and the second position may be a movement in axial and/or rotational and/or radial direction.

The mechanism unit may include a control unit for operating the actuator. The control unit may include a processor and/or an IC chip. The control unit may be a micro-controller. For operating the actuator, the control unit may send an electrical signal.

The actuator may be provided with an electrical signal or electrical current. Without an electrical signal/current, the actuator element may remain in the first position. When the actuator element is in the second position and the actuator is no longer operated or provided with an electrical signal/current, the actuator element may automatically return into the first position. For this purpose, the actuator element in the second position may be pre-biased towards the first position. In other words, the actuator element may be in the first position by default and may only leave the first position when the actuator is operated.

Alternatively, without being provided with an electrical signal/current, the actuator element may be in the second position. When the actuator element is in the first position and the actuator is no longer provided with an electrical signal/current, the actuator element may automatically return into the second position.

According to some embodiments, the mechanism unit is configured to be operatively coupled with a drug reservoir unit.

The drug reservoir unit may include or may be a drug reservoir and/or a drug reservoir holder for holding a drug reservoir. The drug reservoir holder may be configured to hold the drug reservoir such that the drug reservoir is not movable relative to the drug reservoir holder. The drug reservoir may be a cartridge connectable with an injection needle or may be a syringe including an injection needle. The drug reservoir may include a drug, e.g., several doses of a drug.

The drug reservoir may have a distal end for dispensing the drug. The distal end may be the end including a needle or the end to be connected with a needle. The drug reservoir may include a stopper sealing the drug reservoir in proximal direction.

“Operatively coupled” particularly means that the mechanism unit and the drug reservoir unit are mechanically coupled or connected, e.g., releasably coupled or connected. For this purpose, the mechanism unit may include an interface feature for forming a connection interface connecting the mechanism unit to the drug reservoir unit. The interface feature may include a thread configured to engage into a thread of the drug reservoir unit for forming the connection interface. Alternatively, the interface feature may be configured to establish a snap connection to the drug reservoir unit. When coupled, the drug reservoir unit may be fixed with respect to the housing element so that, e.g., the drug reservoir unit is not movable with respect to the housing element in an axial direction. Additionally, or alternatively, “operatively coupled” may mean that the mechanism unit and the drug reservoir unit are coupled to exchange information, e.g., electrical signals or currents.

According to some embodiments, the mechanism unit is configured to enable a dispense process for dispensing a drug dose, e.g., a set drug dose. Particularly, the mechanism unit may be configured to act on a drug reservoir, particularly a drug reservoir of the drug reservoir unit, during the dispense process. When the mechanism unit acts on the drug reservoir, the mechanism unit may push the stopper in distal direction to dispense a drug dose. For example, the plunger rod of the mechanism unit thereby abuts against the stopper and pushes the stopper in distal direction. Performing the dispense process may require the user to operate the dose dispense member.

According to some embodiments, the mechanism unit is configured to enable setting a drug dose to be dispensed. In other words, the mechanism unit may be configured to enable a setting process for setting a drug dose to be dispensed. For example, the drug delivery device is a variable dose device where different drug doses can be set or dialed, respectively, by the user. Setting a drug dose may require a user to operate the dose setting member.

According to some embodiments, setting a drug dose is associated with a movement of the first movable element in a first direction. For example, a drug dose cannot be set without a movement of the first movable element in the first direction. The first direction may be an axial and/or rotational and/or radial direction.

According to some embodiments, the mechanism unit is configured to block movement of the first movable element in the first direction when the actuator element is in the first position to prevent setting a drug dose. This operational state of the mechanism unit is herein also called locked state.

By way of example, the actuator element in the first position is configured to block movement of the first movable element. In this case, a block interface may be formed between the actuator element in the first position and the first movable element. Alternatively, to block movement of the first movable element, a block interface can be formed between the first movable element and an intermediate element different from the actuator element. The actuator element in the first position may hold the intermediate element in a lock position in which the block interface is established.

According to some embodiments, the mechanism unit is configured to allow movement of the first movable element in the first direction when the actuator element is in the second position. Movement of the first movable element in the first direction is, in particular, a precondition for setting a drug dose. For example, when the actuator element is in the second position, dose setting is enabled. Alternatively, the mechanism unit may include a further blocking mechanism to prevent dose setting and only when the actuator element is in the second position and the further blocking mechanism is released, dose setting is enabled. The operational state of the mechanism unit in which setting a drug dose is enabled is herein also called unlocked state.

In some embodiments, the drug delivery device includes a mechanism unit with a housing element, a first movable element arranged movable with respect to the housing element and an electromechanical actuator which, when operated, moves an actuator element between a first position and a second position. The mechanism unit is configured to be operatively coupled with a drug reservoir unit and to enable a dispense process for dispensing a drug dose. Furthermore, the mechanism unit is configured to enable setting a drug dose to be dispensed, where setting a drug dose is associated with a movement of the first movable element in a first direction. Moreover, the mechanism unit is configured to block movement of the first movable element in the first direction when the actuator element is in the first position to prevent setting a drug dose and to allow movement of the first movable element in the first direction when the actuator element is in the second position as a precondition for setting a drug dose.

A mechanism unit with an electromechanical actuator used for blocking or releasing dose setting makes the drug delivery device safer because setting a drug dose may be tied to conditions, for example that a selected drug reservoir unit is coupled to the mechanism unit, said drug reservoir unit including a prescribed drug, or that the user of the drug delivery device is authorized to set a dose.

The drug delivery device specified herein may be elongated and/or may include a longitudinal axis, e.g., a main extension axis. Additionally, or alternatively, the drug delivery device may have a rotational symmetry with respect to the longitudinal axis. A direction parallel to the longitudinal axis is herein called an axial direction. By way of example, the drug delivery device may be cylindrically shaped.

Furthermore, the drug delivery device may include an end, e.g., a longitudinal end, which may be provided to face or to be pressed against a skin region of a human body. This end is herein called the distal end. A drug or medicament may be supplied via the distal end. The opposing end is herein called the proximal end. The proximal end is, during usage, remote from the skin region. The axial direction pointing from the proximal end to the distal end is herein called distal direction. The axial direction pointing from the distal end to the proximal end is herein called proximal direction. A distal end of a member or element or feature of the drug delivery device is herein understood to be the end of the member/element/feature located most distally.

Accordingly, the proximal end of a member or element or feature is herein understood to be the end of the element/member/feature located most proximally.

In other words, distally is used herein to specify directions, ends or surfaces which are arranged or are to be arranged to face or point towards a dispensing end of the drug delivery device or components thereof and/or point away from, are to be arranged to face away from or face away from the proximal end. On the other hand, proximal is herein used to specify directions, ends or surfaces which are arranged or are to be arranged to face away from or point away from the dispensing end and/or from the distal end of the drug delivery device or components thereof. The distal end may be the end closest to the dispensing end and/or furthest away from the proximal end and the proximal end may be the end furthest away from the dispensing end. A proximal surface may face away from the distal end and/or towards the proximal end and a distal surface may face towards the distal end and/or away from the proximal end. The dispensing end may be a needle end where a needle unit is or is to be mounted to the device, for example.

A direction perpendicular to the longitudinal axis and/or intersecting with the longitudinal axis is herein called radial direction. An inward radial direction is a radial direction pointing towards the longitudinal axis. An outward radial direction is a radial direction pointing away from the longitudinal axis. The term “angular direction,” “azimuthal direction” or “rotational direction” are herein used as synonyms. Such a direction is a direction perpendicular to the longitudinal axis and perpendicular to the radial direction.

According to some embodiments, the mechanism unit is configured to prevent dispensing a drug dose when the actuator element is in the first position. Additionally, or alternatively, the mechanism unit may be configured to enable dispensing a drug dose when the actuator element is in the second position.

Alternatively, the mechanism unit may be configured to enable dispensing a drug dose, irrespectively of the position of the actuator element.

According to some embodiments, dispensing a drug dose is associated with a movement of the first movable element in a second direction. The second direction may be different from the first direction, e.g., opposite to the first direction. For example, dispensing of a drug dose is not possible without a movement of the first movable element in the second direction.

According to some embodiments, the mechanism unit is configured to block movement of the first movable element in the second direction when the actuator element is the first position. Additionally, or alternatively, the mechanism unit may be configured to allow movement of the first movable element in the second direction when the actuator element is in the second position. Movement of the first movable element in the second direction may be a precondition for dose dispensing.

Alternatively, the mechanism unit may be configured to allow movement of the first movable element in the second direction, irrespectively of the position of the actuator element.

According to some embodiments, the mechanism unit is configured such that, unless a selected drug reservoir unit is coupled with the mechanism unit, operation of the actuator is prevented. A selected drug reservoir unit is, in particular, a drug reservoir unit which is especially intended or foreseen or selected for the mechanism unit. For example, as long as no selected drug reservoir unit is coupled to the mechanism unit, the actuator element remains in the first position. For example, a selected drug reservoir unit is a drug reservoir unit with an electrical contact element at a correct or predefined position.

According to some embodiments, the mechanism unit is configured such that a coupling of the mechanism unit to a selected drug reservoir unit is a precondition for the operation of the actuator and/or for moving the actuator element out of the first position into the second position. For example, only when the mechanism unit is coupled with a selected drug reservoir unit, operation of the actuator is enabled, or the actuator is automatically operated. If the mechanism unit is not coupled to a selected drug reservoir unit, operation of the actuator may be disabled.

According to some embodiments, the mechanism unit includes a first conductor path. The first conductor path may include metal. For example, the first conductor path is interrupted, e.g., not closed, unless a selected drug reservoir unit is coupled with the mechanism unit.

According to some embodiments, the first conductor path includes at least one contact point for electrically contacting at least one contact element of a drug reservoir unit. The contact point may be arranged such that, at least unless the mechanism unit is coupled to a drug reservoir unit, the contact point is freely accessible. The contact point may be an electrically conductive area of the mechanism unit. The contact point may be arranged at a distal end of the mechanism unit and/or may face in distal direction.

For example, the first conductor path includes two contact points for electrically contacting the contact element. The first conductor path may be interrupted between the two contact points. The two contact points may be spaced from each other in rotational direction. The two contact points may overlap or may be aligned in axial and/or radial direction.

According to some embodiments, when a selected drug reservoir unit with a contact element at a correct position is coupled with the mechanism unit, the at least one contact point electrically contacts the contact element and this changes an electrical property of the first conductor path in a characteristic manner. Particularly, the electrical resistance of the first conductor path may be changed in a characteristic manner in this way.

By way of example, if an unselected drug reservoir unit is coupled with the mechanism unit with no contact element at the correct position, the electrical property of the first conductor path is not changed in a characteristic manner or is not changed at all.

For example, a selected drug reservoir unit includes a contact element with at least one access point, e.g., two access points. The access points may be electrically conductive areas of the contact element. The access points may constitute ends of the contact element. The access points may be electrically connected via the contact element. The access points may be arranged at a proximal end of the drug reservoir unit and/or may face in proximal direction. A selected drug reservoir unit may include a contact element with at least one access point which, when the drug reservoir unit is coupled with the mechanism unit, faces and contacts the at least one contact point. For example, every access point then faces and electrically contacts a different contact point of the mechanism unit. For example, the access points of a selected drug reservoir unit overlap or are aligned in rotational direction with the contact points when the drug reservoir unit and the mechanism unit are coupled.

According to some embodiments, the mechanism unit is configured such that operation of the actuator is prevented and/or movement of the actuator element out of the first position into the second position is prevented unless the electrical property of the first conductor path is changed in at least one characteristic manner.

According to some embodiments, when a selected drug reservoir unit is coupled with the mechanism unit, the first conductor path is closed. Closing the first conductor path may be a change of an electrical property of the first conductor path in a characteristic manner. When no drug reservoir unit is coupled with the mechanism unit or an unselected drug reservoir unit is coupled with mechanism unit, the first conductor path may not be closed. The first conductor path may, in particular, be closed by the contact element of the selected drug reservoir unit.

According to some embodiments, the closed first conductor path electrically connects components of the mechanism unit. For example, only when the first conductor path is closed, the actuator is operable, or operation of the actuator is enabled.

According to some embodiments, the closed first conductor path electrically connects the actuator with a control unit of the mechanism unit. Additionally, or alternatively, the closed first conductor path may electrically connect the control unit with an energy source of the mechanism unit and/or may electrically connect the actuator with the energy source and/or electrically connects an output interface of the control unit with an input interface of the control unit. For example, if the first conductor path is not closed, the mentioned components may not be electrically connected.

By way of example, when the first conductor path is closed due to a coupling of the mechanism unit with a selected drug reservoir unit, the control unit can send a test signal via the output interface along the closed first conductor path. The control unit may be configured to receive the test signal via the input interface. The control unit may be configured to operate the actuator based on or in response of the received test signal. For example, only when the control unit receives the test signal, the actuator is operated.

Another possibility is that only when the first conductor path is closed, the control unit is electrically connected to an energy source and only then is provided with energy. Yet another possibility is that, when the first conductor path is closed, the energy source is electrically connected to the actuator and the actuator is then automatically operated without additional operation signals of a control unit.

According to some embodiments, the first conductor path includes at least two sections which are arranged movable with respect to each other. The two sections may be electrically connected by a sliding contact.

According to some embodiments, the two sections are arranged rotatably and/or axially movable with respect to each other.

The two sections may be assigned to different elements of the mechanism unit, e.g., are arranged on different elements of the mechanism unit. The different elements may be arranged movable with respect to each other. For example, the different elements move with respect to each other during an operation of the mechanism unit so that also the two sections of the first conductor path move with respect to each other. The operation of the mechanism unit during which the elements move relative to each other may be a dose setting process and/or a dose dispense process. For example, the two sections move axially and/or rotationally with respect to each other during this operation. By way of example, one section of the first conductor path is assigned to the first movable element.

According to some embodiments, during an operation of the mechanism unit, a first section of the first conductor path moves on a helical path with respect to a second section of the first conductor path.

According to some embodiments, the first section includes a helical conductor track. The helical conductor track may be electrically connected to the second section via the sliding contact.

According to some embodiments, the helical conductor track has the same pitch as the helical path so that during operation of the mechanism unit, the two sections of the first conductor path stay electrically connected.

According to some embodiments, the mechanism unit further includes a communication module for communicating with an external device. The external device may include a processor. For example, the external device is a computer or a smartphone or a smartwatch.

The communication module may be electrically coupled with the control unit. The communication module may be configured for a wireless communication with the external device, e.g., for a Bluetooth communication.

According to some embodiments, the mechanism unit is configured such that operation of the actuator is prevented unless an enabling signal from the external device is received via the communication module. The communication module may then transmit the enabling signal to the control unit and the control unit may, in response to the enabling signal, send an operation signal to the actuator to operate the actuator.

For example, only if the electrical property of the first conductor path is changed in at least one characteristic manner and only if the enabling signal is received, operation of the actuator is enabled or only then the actuator is operated.

For example, the external device may first be used to identify the drug reservoir unit coupled with the mechanism unit, particularly to identify the drug of the drug reservoir unit. For this purpose, the drug reservoir unit may include a code, like a QR code, which is characteristic for the drug reservoir unit or the kind of the drug reservoir unit. The QR code may be read with the external device to identify the drug reservoir unit. The external device may then be configured to decide whether the drug reservoir unit is a correct drug reservoir unit for the user of the external device, e.g., including a prescribed drug. Only if this is the case, the enabling signal may be sent by the external device.

In this way, one mechanism unit can be used for different kinds of selected drug reservoir units. However, the operation of the actuator to allow setting of a drug dose is enabled only if a selected drug reservoir unit is coupled with the mechanism unit and only if this selected drug reservoir unit is indeed intended for the user. This may further increase the safety for the user.

Different kinds of selected drug reservoir units may, e.g., differ from each other by the position of their contact element, particularly by the position, e.g., the offset in rotational direction, of the respective access points. The mechanism unit may include a different first conductor path for every kind of selected drug reservoir unit with contact points at respective positions.

According to some embodiments, the first movable element is rotated and/or moved axially when being moved in the first direction during setting a drug dose. For example, the first movable element is moved on a helical path during dose setting.

According to some embodiments, the mechanism unit further includes an intermediate element which is displaceable between a lock position and a release position. The intermediate element is, in particular, different from the actuator element and/or the first movable element.

According to some embodiments, movement of the actuator element from the first position into the second position enables a movement of the intermediate element from the lock position into the release position and/or vice versa.

For example, when the actuator element moves from the first position into the second position, the intermediate element automatically moves from the lock position into the release position. For this purpose, the intermediate element in the lock position may be pre-biased towards the release position.

Alternatively, when the actuator element moves from the second position into the first position, the intermediate element may automatically move from the release position into the lock position. For this purpose, the intermediate element in the release position may be pre-biased towards the lock position.

According to some embodiments, the intermediate element in the lock position is configured to block movement of the first movable element in the first direction. Particularly, the block interface is then formed between the intermediate element in the lock position and the first movable element.

According to some embodiments, a path along which the actuator element moves between the first and the second position is different from a path along which the intermediate element moves between the lock position and the release position. For example, a path or a direction of the path along which the actuator element moves is perpendicular or at least partially perpendicular to a path or a direction of the path along which the intermediate element moves.

According to some embodiments, the drug delivery device includes a dose setting member configured to be operated by a user to set a drug dose. The dose setting member may be the above-mentioned user interface member, particularly may be a knob. The dose setting member is, in particular, configured to be touched by a user for setting a drug dose.

According to some embodiments, during dose setting, the dose setting member is moved relative to the housing element. For example, the dose setting member is moved on a helical path in proximal direction relative to the housing element.

According to some embodiments, an energy source and/or a control unit for operating the actuator and/or the actuator element are moved during setting a drug dose. For example, the energy source and/or the control unit and/or the actuator element are coupled to the dose setting member and move together with the dose setting member. The energy source and/or the control unit may be fixed to the dose setting member, e.g., axially and/or rotationally. Alternatively, the energy source and/or the control unit may be fixed, e.g., axially and/or rotationally to the setting element (see above, e.g., formed by the number sleeve or dial sleeve). The dose setting member and the setting element may be rotationally and/or axially locked to each other during dose setting (e.g., during the setting process). During dose delivery (e.g., during the dispense process) relative rotational (and/or relative axial) movement between the setting element and the dose setting member may be possible. Thus, during dose delivery, the energy source and/or the control unit may rotate relative to the dose setting member (or user interface member) or not.

According to some embodiments, the mechanism unit further includes a second conductor path for guiding an electrical signal to the actuator. For example, the second conductor path electrically connects the actuator with the control unit and/or the battery.

According to some embodiments, the second conductor path includes at least two sections which are arranged movable with respect to each other, and which are electrically connected by sliding contact.

All features disclosed in connection with the first conductor path are also disclosed for the second conductor path and vice versa. Particularly, one section of the second conductor path may include a helical conductor track.

According to some embodiments, the actuator includes a magnet and an electromagnet. The electromagnet may be coupled or fixed to the actuator element, and the magnet may be coupled or fixed to a further element of the mechanism unit. The magnet may be fixed with respect to the housing element, or vice versa.

According to some embodiments, a magnetization of the electromagnet is changed when the actuator is operated.

According to some embodiments, magnetic interaction between the magnet and the electromagnetic results in movement of the actuator element between the first position and the second position when the actuator is operated. For example, the magnetic interaction alone is responsible for the movement of the actuator element when the actuator is operated. The interaction between the electromagnet and the magnet may be changed from attractive to repellant or vice versa when the actuator is operated.

Additionally, or alternatively, the magnetic interaction between the magnet and the electromagnet may hold the actuator element in the first position, e.g., as long as the actuator is not operated. When the actuator is operated, the magnetic interaction may be turned off. The actuator element may then automatically move into the second position due to a pre-bias towards the second position.

According to some embodiments, the mechanism unit is configured to block movement of the first movable element in the first direction when the actuator element is in the first position and when no drug dose is set and/or when a drug dose is already set. For example, the mechanism unit is configured to block movement of the first movable element in the first direction when the actuator element is in the first position at different positions of the first movable element. Likewise, the mechanism unit may be configured to block movement of the first movable element in the second direction when no dose is set and/or when a drug dose is already set.

According to some embodiments, the actuator element is movable in radial direction between the first position and the second position. Additionally, or alternatively, the actuator element may be movable in an axial direction and/or in rotational direction between the first position and the second position. When the actuator element is movable in radial, axial, or rotational direction between the first and the second position, the magnets may be arranged opposite each other in radial, axial, or rotational direction, respectively, and may be arranged to overlap in the respective other two directions.

According to some embodiments, the actuator element is elongated. The actuator element may, e.g., orientated, in axial and/or in rotational direction. This particularly means that the actuator element may extend along the axial and/or rotational direction.

According to some embodiments, the actuator element is an arm. One end of the arm may be a free end which is displaceable. The other end of the arm may be fixed to an element of the mechanism unit, e.g., to the housing element or to an element fixed with respect to the housing element. The magnet assigned to the arm may be located at the free end, e.g., closer to the free end than to the other longitudinal end.

According to some embodiments, the actuator element and the first movable element are configured to engage with each other when the actuator element is in the first position. For example, one of the actuator elements and the first movable element includes a protrusion and the other one of the actuator element and the first movable element includes a recess. When engaged, the protrusion projects into the recess. Blocking of movement of the first movable member in the first and/or second direction may be due to an abutment between the protrusion and an inner surface delimiting the recess.

Likewise, if an intermediate element is used, the intermediate element and the first movable element may be configured to engage with each other when the intermediate element is in the lock position.

According to some embodiments, the actuator includes an electric motor for moving the actuator element.

According to some embodiments, the actuator element is coupled to the first movable element. Particularly, movement of the first movable element in the first and/or second direction may result in movement of the actuator element in the same direction.

According to some embodiments, the intermediate element and/or the actuator element are configured to engage the housing element or an element which is fixed with respect to the housing element when in the lock position or first position, respectively.

According to some embodiments, the actuator includes a spindle. The actuator element may be a spindle nut which is moved by the spindle.

According to some embodiments, the intermediate element moves radially and/or axially between the release position and the lock position. For example, the intermediate element may move in an axial direction and radial outward direction when being moved from the release position into the lock position.

According to some embodiments, the drug delivery device includes a drug reservoir unit coupled with the mechanism unit. The drug reservoir unit may include a drug reservoir filled with a drug and/or a drug reservoir holder. The drug reservoir unit may be a selected drug reservoir unit.

According to some embodiments, the actuator element is a displaceable or movable element. The displaceable or movable element may be in the form of a flexible arm. The flexible arm may include an electromagnet, e.g., at the free longitudinal end thereof.

According to some embodiments, the flexible arm is orientated in an axial direction. According to another embodiment, the flexible arm is orientated in a circumferential direction. According to some embodiments, the actuator includes an actuator element in the form of an elliptical disc.

Hereinafter, the drug delivery device will be explained in more detail with reference to drawings on the basis of exemplary embodiments. Same reference signs indicate similar, similar acting or same elements in the individual Figures. However, the size ratios involved are not necessarily to scale, individual elements may be illustrated with exaggerated size for better understanding.

### BRIEF DESCRIPTION OF THE FIGURES

FIGS. 1 through 9 show a first exemplary embodiment of a drug delivery device in different views.

FIGS. 10 through 13 show a second exemplary embodiment of a drug delivery device in different views.

FIGS. 14 through 17 show a third exemplary embodiment of a drug delivery device in different views.

FIGS. 18 through 21 show a fourth exemplary embodiment of a drug delivery device in different views.

FIGS. 22 through 25 show a fifth exemplary embodiment of a drug delivery device in different views.

FIGS. 26 through 28 show a sixth exemplary embodiment of a drug delivery device in different views.

FIG. 29 shows an exemplary embodiment of a drug delivery device in a cross-sectional view.

FIG. 30 shows exemplary embodiments of a drug reservoir unit each in a cross-sectional view.

FIGS. 31 and 32 show sections of the second exemplary embodiment of the drug delivery device in different states.

### DETAILED DESCRIPTION

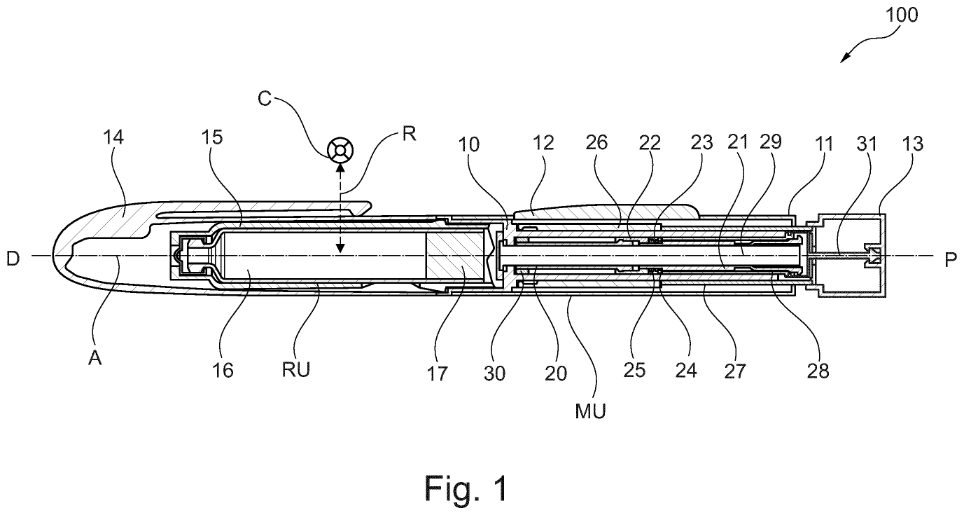

FIG. 1 shows a first exemplary embodiment of a drug delivery device 100 in a cross-sectional view. The drug delivery device 100 is a variable dose device, in which different doses of a drug to be dispensed can be set or dialed, respectively, by a user. The drug delivery device is a dial extension pen.

FIG. 1 also indicates the coordinate system used herein for specifying positions of members or elements or features. The distal direction D and the proximal direction P run parallel to the longitudinal axis A. The longitudinal axis A is a main extension axis of the device 100. The radial direction R is a direction perpendicular to the longitudinal axis A and intersecting with the longitudinal axis A. The azimuthal direction C, also referred to as angular direction or rotational direction, is a direction perpendicular to the radial direction R and to the longitudinal axis A. The different directions and axes are not indicated in the following Figures.

The drug delivery device 100 includes a mechanism unit MU with a setting mechanism and a dispense mechanism. The setting mechanism is configured for setting a drug dose and the dispense mechanism is configured for dispensing a drug dose. The functional principles of the mechanisms are explained below.

The mechanism unit MU includes an inner body 10 and a housing element, in the following also referred to as outer body 11. The inner body 10 and the outer body 11 are fixedly connected to each other, e.g., they cannot be rotated or moved axially with respect to each other. The outer body 11 forms an outer surface of the drug delivery device 100 which can be touched or grabbed by a user.

The drug delivery device 100 further includes a cap 14 and a user interface member in the form of a knob 13. The knob 13 is a dose setting member configured to be operated by a user for setting a drug dose. At the same time, the knob 13 is dose dispense member configured to be operated by a user to dispense a drug dose.

A drug reservoir unit RU including a reservoir 16 and a reservoir holder 15 is received within the cap 14. A drug is filled into the reservoir 16. The reservoir 16 is sealed in the proximal direction P by a stopper 17.

The drug reservoir unit RU is operatively coupled or connected, respectively, to the mechanism unit MU. The mechanism unit MU is configured to enable a dispense process for dispensing a drug dose by acting on the drug reservoir 16. For dispensing a drug dose, the stopper 17 is pushed in the distal direction D by a plunger rod 29 of the mechanism unit MU. The coupling between the mechanism unit MU and the reservoir unit RU is realized by the inner body 10 being coupled to the reservoir holder 15 via a connection interface which might be a snap connection or a threaded connection. The coupling is preferably reversible. For example, the drug reservoir unit RU is axially and rotationally fixed to the inner body 10 by the coupling.

The mechanism unit MU further includes a number sleeve 26 and a dial sleeve 27 which are fixedly coupled to each other (e.g., they cannot rotate or move axially relative to each other). The dial sleeve 27 and number sleeve 26 can be implemented by one unitary component. Hence, referrals herein to the number sleeve should be considered as referrals to the dial sleeve and vice versa. The number sleeve 26 may include an inner thread which is engaged with an outer thread of the inner body 10. On an outer surface of the number sleeve 26, numbers may be shown, e.g., suitable to indicate the size of the currently set dose. The user can see the numbers through a window 12 of the mechanism unit MU. The window 12 may include a lens. The window 12 is formed in the outer body 11. The numbers visible in the window 12 indicate to a user the set/dialed dose. Due to the threaded coupling between the number sleeve 26 and the inner body 10, the dial sleeve 27 and the number sleeve 26 are moved on a helical path in proximal direction relative to the body 10, 11 during setting a drug dose and dispensing a drug dose as explained below.

The mechanism unit MU also includes a drive sleeve. The drive sleeve includes a distal drive sleeve 20, a proximal drive sleeve 21 and a drive sleeve coupler 22 coupling the distal drive sleeve 20 to the proximal drive sleeve 21. For setting a drug dose and dispensing a drug dose, the distal drive sleeve 20 and the proximal drive sleeve 21 are fixedly coupled to each other via the drive sleeve coupler 22 so that these elements can neither rotated nor move axially relative to each other during setting and dispensing of a dose. The distal drive sleeve 20 may include an inner thread which is engaged with an outer thread of the plunger rod 29. An outer thread of the distal drive sleeve 20 may be engaged to an inner thread of a last dose nut 30, the function of which will be explained below. The distal drive sleeve and the proximal drive sleeve may be uncoupled, e.g., for a resetting operation when the plunger rod should be moved back into an initial position to reuse the mechanism unit MU for a new reservoir. The uncoupling for the reset, e.g., by moving teeth of the distal and proximal drive sleeves out of engagement, may achieve that the distal drive sleeve can rotate relative to the proximal drive sleeve, thereby enabling a movement of the plunger rod into an initial position. Thus, the drug delivery device can be a reusable device.

Furthermore, the mechanism unit MU includes a clutch 28, which is fixedly coupled to the knob 13 so that, during setting a drug dose and dispensing a drug dose, the clutch 28 and the knob 13 are not rotated or moved axially relative to each other. A clutch coupler 31 may be provided for this purpose. The clutch coupler 31 expediently rotationally and/or axially locks the knob 13 and the clutch 28 with one another. Clutch 28 and knob 13 could also be integrally formed. Different couplings between clutch and knob than the depicted clutch coupler 31 are possible. The clutch coupler 31 has portions of different outer diameters. In a first portion, the clutch coupler may be connected or engaged to the clutch 28. For example, an inner surface of the clutch coupler 31 may extend along an outer surface of the clutch 28. The clutch 28 or a portion thereof may be received within the first portion of the clutch coupler. A second portion, which may project from the first portion in a central region of the first portion and/or extend proximally, e.g., towards the proximal end of the knob, has an outer diameter which is less than the outer diameter of the first portion. The second portion may have a rod-like configuration. In the second portion, the clutch coupler may extend through an opening in an element is provided within the knob 13 and/or on the dial sleeve 27. This element may be or may include a conductor carrier or circuit board (not shown in FIG. 1). The clutch 28 is coupled to the proximal drive sleeve 21 via a splined engagement. This splined engagement may allow a certain axial movement of the clutch 28 relative to the proximal drive sleeve 21 but does not allow a relative rotation between these two elements.

A distal clicker 23, a proximal clicker 24 and a clutch spring 25 of the mechanism unit MU are arranged between the clutch 28 and the drive sleeve coupler 22. The clutch spring 25 is coupled to the drive sleeve coupler 22 and to the distal clicker 23. The distal clicker 23 is configured to engage the proximal clicker 24 in the proximal direction P. The distal clicker and the proximal clicker can be configured to be coupled via a toothed interface, e.g., via engageable sets of circumferentially disposed teeth (which may be provided at the inner radius or circumference of the clickers 23, 24). The toothed interface may enable rotation of one of the clickers relative to the other one of the clickers under simultaneous axial displacement with the clickers 23 and 24 being biased into engagement via the clutch spring 25 (thereby providing a clicking noise by the rotating teeth). The proximal clicker 24 is configured to abut against the clutch 28 in the proximal direction P. Thus, the clutch spring 25 is configured to bias the distal clicker 23, the proximal clicker 24 and the clutch 28 in the proximal direction P relative to the drive sleeve coupler 22. The mechanism of the device described herein operates like the device disclosed in WO 2015/028441 A1, the entire disclosure of which is incorporated herein by reference. The dial sleeve and the number sleeve as well as the remaining parts of the mechanism are illustrated slightly differently in the Figures of the present application, but may nevertheless be implemented as depicted and/or described in WO 2015/028441 A1.

The distal clicker 23 may be permanently splined to the proximal drive sleeve 21 so that a relative rotation between these two elements is prevented. However, a certain axial movement between the distal clicker 23 and the proximal drive sleeve 21 may be allowed. The proximal clicker 24 may be permanently splined to the inner body 10 so that a relative rotation between these two elements is prevented, whereas a certain relative axial movement is allowed.

The distal face of the clutch 28 and the proximal face of the proximal clicker 24 may both be toothed so that these two faces may engage into each other. Furthermore, the distal face of the proximal clicker 24 and the proximal face of the distal clicker 23 may both be toothed so that these two toothed faces can engage into each other. A proximal face of the clutch 28 may be toothed, e.g., dog toothed, and may be arranged to engage a toothed, e.g., dog toothed, distal face of the dial sleeve 27.

FIG. 1 shows the drug delivery device 100 when no dose is set (0 units/0-unit position). Dose setting may be allowed in discrete units of 1, e.g., from 0 to 80 units. For setting a desired drug dose, the user has to rotate the knob 13. This is done without pressing on the knob 13 in the distal direction D. As long as one does not press on the knob 13 in the distal direction D, a dog toothed engagement between the clutch 28 and the dial sleeve 27 is established due to the clutch spring 25 either biasing the clutch 28 in the proximal direction P or at least preventing the clutch 28 from moving in the distal direction D. The dog toothed engagement between the clutch 28 and the dial sleeve 27 has as a consequence that the two elements are rotationally locked to each other so that, when the knob 13 is rotated, also the dial sleeve 27 and the number sleeve 26 are rotated. Since the number sleeve 26 is threadedly engaged with the inner body 10, rotating the knob 13 has as a consequence that the knob 13, the clutch 28, the dial sleeve 27 and the number sleeve 26 move on a helical path in the proximal direction P relative to the body 10, 11. Thereby, the numbers of the number sleeve 26 visible through the window 12 increase, e.g., as the set dose increases.

Since the proximal drive sleeve 21 is splined to the clutch 28, the proximal drive sleeve 21 as well as the distal drive sleeve 20 and the drive sleeve coupler 22 are moved on the helical path in the proximal direction P relative to the inner body 10.

The plunger rod 29 includes two outer threads with opposite hand which overlap with each other. The plunger rod 29 is threadedly engaged with the inner thread of the distal drive sleeve 20. The threads are chosen such that during the helical movement of the distal drive sleeve 20 in the proximal direction P, the plunger rod 29 does not rotate and is also not moved axially.

The last dose nut 30 may be splined to the inner body 10 and, therefore, cannot rotate relative to the inner body 10. Due to the threaded engagement of the last dose nut 30 with the distal drive sleeve 20, the last dose nut 30 is forced to move in the proximal direction P during setting a drug dose. When the maximum dose has been set (e.g., 80 units—independently of whether the dose has been set in one drug setting process or several drug setting processes), the last dose nut 30 establishes a rotation-lock interface with the distal drive sleeve 20 so that the last dose nut 30 can no longer rotate relative to the distal drive sleeve 20. As a consequence of this, the distal drive sleeve 20 can no longer be rotated and no further drug dose can be set. The drug delivery device 100 then has to be reset to an initial state.

During setting a drug dose, the toothed faces of the distal clicker 23 and the proximal clicker 24 facing each other ratchet over each other thereby creating a click sound which indicates to a user that a drug dose is set. For this purpose, the teeth of the two faces are preferably formed as shallow triangles so that relative rotation between the clickers 23 and 24 is possible, leading to a repeated slight compression and decompression of the clutch spring 25.

After the desired dose has been set, the user can now press on the knob 13 in the distal direction D to dispense the set drug dose. Thereby, the distally directed force on the knob 13 is transferred from the knob 13 via the clutch 28 to the proximal clicker 24, from there to the distal clicker 23 and this compresses the clutch spring 25. The two clickers 23 and 24 are now pressed against each other and their toothed faces are engaged. When the knob 13 is pressed in the distal direction, the proximal clicker 24 is expediently brought into a splined connection with the proximal drive sleeve 21 to which the distal clicker 23 is permanently splined already. Hence the proximal drive sleeve 21 may be splined to both clickers when the knob 13 is pressed. Relative rotation between the two clickers 23, 24 is thus prevented. Since the proximal clicker 24 is splined to the inner body 10 and the distal clicker 23 is splined to the proximal drive sleeve 21, the proximal drive sleeve 21 can no longer rotate relative to the inner body 10. However, since the proximal drive sleeve 21 is also splined to the clutch 28, the clutch 28 and the knob 13 can no longer rotate relative to the inner body 10.

The distally directed force applied to the knob 13 has as a consequence that the clutch 28 together with the knob 13 slightly moves in the distal direction D relative to the dial sleeve 27 so that the clutch spring 25 is compressed, as already mentioned. The dog toothed engagement between the dial sleeve 27 and the clutch 28 is thereby released so that the dial sleeve 27 is no longer rotationally locked to the clutch 28. Therefore, when the knob 13 is pressed in the distal direction D, the dial sleeve 27 together with the number sleeve 26 can still rotate relative to the inner body 10. When the knob 13 is now moved in the distal direction D, a stop against the dial sleeve 27 forces the dial sleeve 27 to also move in the distal direction D. Due to the threaded engagement of the number sleeve 26 with the inner body 10, the dial sleeve 27 together with the number sleeve 26 moves on a helical path in the distal direction D. Thereby, the numbers of the number sleeve 26 visible in the window 12 decrease.

At the same time, the clutch 28, the clickers 23, 24 and the drive sleeve 20, 21, 22 are forced to move in the distal direction D (without rotation). The threaded engagement between the plunger rod 29 and the distal drive sleeve 20 forces the plunger rod 29 to rotate. A further threaded engagement between the plunger rod 29 and an inner thread of the inner body 10 may then force the plunger rod 29 to also move distally to push the stopper 17 inside the cartridge in the distal direction D for dispensing the set drug dose. Since the distal drive sleeve 20 is not rotate during dispensing, the last dose not 30 moves together with the distal drive sleeve 20 in the distal direction D without changing position relative to the distal drive sleeve 20.

After having dispensed the set drug dose and when the knob 13 has been completely moved back into an initial position, a new drug dose may be set by again rotating the knob 13 on a helical path in the proximal direction P. During this step, the plunger rod 29 does not change position. When dispensing a dose, the plunger rod 29 is moved in the distal direction D.

As explained with respect to FIG. 1, one user interface member in the form of a knob 13 is used for setting a drug dose as well as for dispensing the drug dose. However, it is also possible to use separate user interface members for setting and dispensing a drug dose.

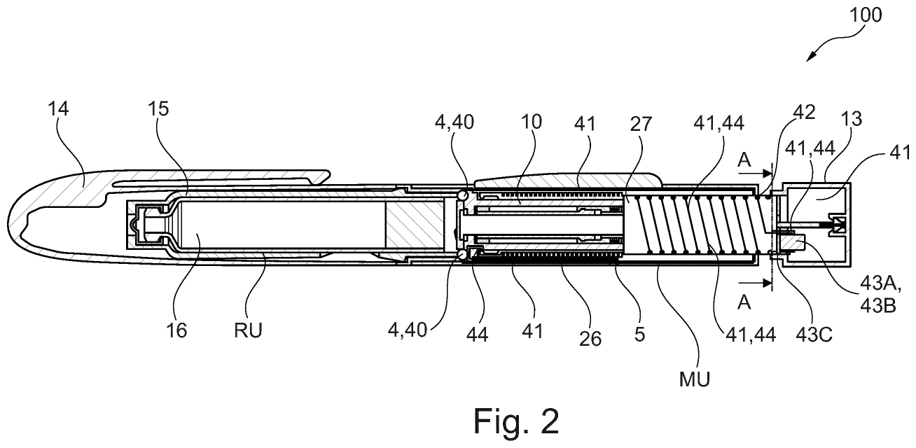

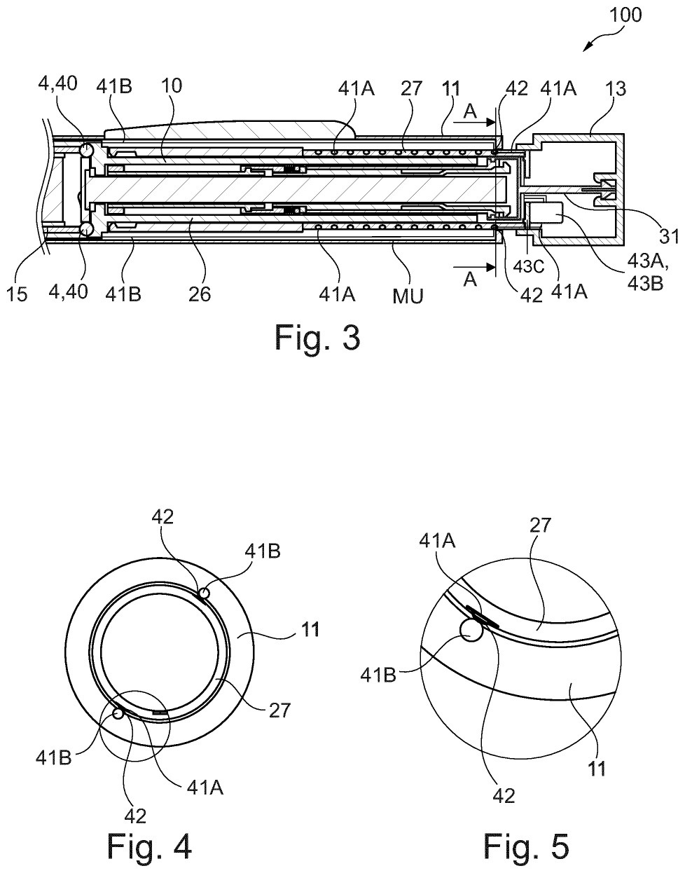

FIGS. 2 and 3 show the drug delivery device 100 of FIG. 1, but in different views than FIG. 1. FIG. 3 shows the proximal part of the drug delivery device 100. As can be seen, the dial sleeve 27 includes a conductor path 41, 44. The conductor path 41, 44 includes a winded or helical conductor track, respectively, which is arranged at the outer surface of the dial sleeve 27. The pitch of the helical conductor track is preferably the same as the pitch of the helical path on which the dial sleeve 27 moves during setting and dispensing a drug dose.

On the proximal face of the dial sleeve 27, a control system including a control unit 43A and a battery 43B are arranged. The control unit 43A and the battery 43B may be arranged on a printable circuit board (PCB) or conductor carrier 43C mounted on the proximal face of the dial sleeve 27. The control unit 43A may include a processor and/or an IC-chip. The control unit 43A and/or the battery 43B may be electrically connected to the conductor path 41, 44. These elements may be mounted on the dial sleeve 27. Hence, they may rotate relative to the knob 13 during the dose delivery operation.

As can be seen in FIG. 3, the conductor path 41 actually includes two sections 41A and 41B. These two sections 41A, 41B are assigned to different elements of the drug delivery device 100. The first section 41A is assigned to the dial sleeve 27 and is fixed to the dial sleeve 27 so as to follow movement of the dial sleeve 27. The second section 41B is assigned to the body 10, 11 and is fixed to the body 10, 11. Therefore, the two sections 41A, 41B move relative to each other during setting a drug dose and dispensing a drug dose.

To always maintain an electrical connection between the first section 41A and the second section 41B during dose setting and dose dispensing, a sliding contact 42 is realized between the two sections 41A, 41B. This sliding contact 42 can be seen in FIGS. 4 and 5. FIG. 4 is a cross-sectional view on the plane AA of FIG. 3 and FIG. 5 is a detailed view showing the circled region of FIG. 4.

The helical conductor track of the first section 41A assigned to the dial sleeve 27 and having the same pitch as the helical path on which the dial sleeve 27 moves relative to the body 10, 11 during dose setting and dose dispensing in combination with the sliding contact 42 ensures that the two sections 41A, 41B always stay electrically connected during dose setting and dose dispensing.

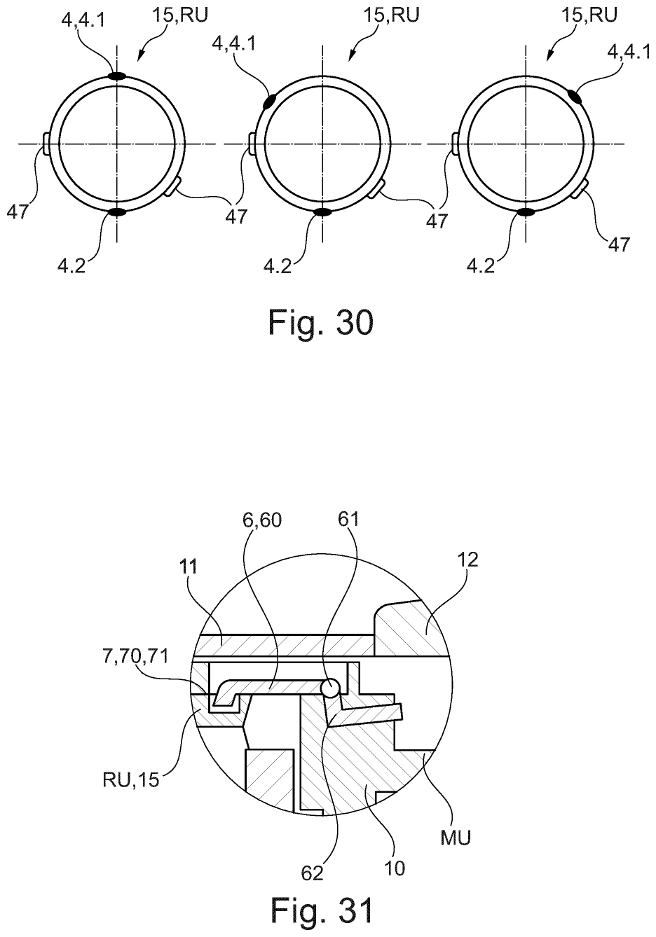

As can be further seen in FIGS. 2 and 3, the second section 41B of the conductor path 41 includes contact points 40 which are configured to electrically connect to a contact element 4 of the drug reservoir unit RU. The contact points 40 are electrically conductive areas, e.g., facing in the distal direction D. When a selected drug reservoir unit RU with a contact element 4 at a correct position, particularly with access points of the contact element 4 at correct positions, is coupled to the mechanism unit MU, the contact points 40 are electrically connected to the contact element 4. This has an influence on an electrical property, namely the electrical resistance, of the conductor path 41. In the present case, the conductor path 41 is then closed by the contact element 4. Further details about the contact element 4 and the contact points 40 are explained in connection with FIGS. 29 and 30.

The closed conductor path 41 may, e.g., electrically connect the control unit 43A to the battery 43B. Alternatively, the control unit 43A may be configured to send an electric test signal via an output interface through the conductor path 41 and only when the conductor path 41 is closed with help of the contact element 4 of the selected drug reservoir unit RU, the test signal is returned to the control unit 43A via an input interface thereof. In this way, the mechanism unit MU can determine that a selected drug reservoir unit RU with a contact element 4 at the correct positions is coupled to the mechanism unit MU. This may then be used to enable a change of the operational state of the mechanism unit MU, which will be explained below.

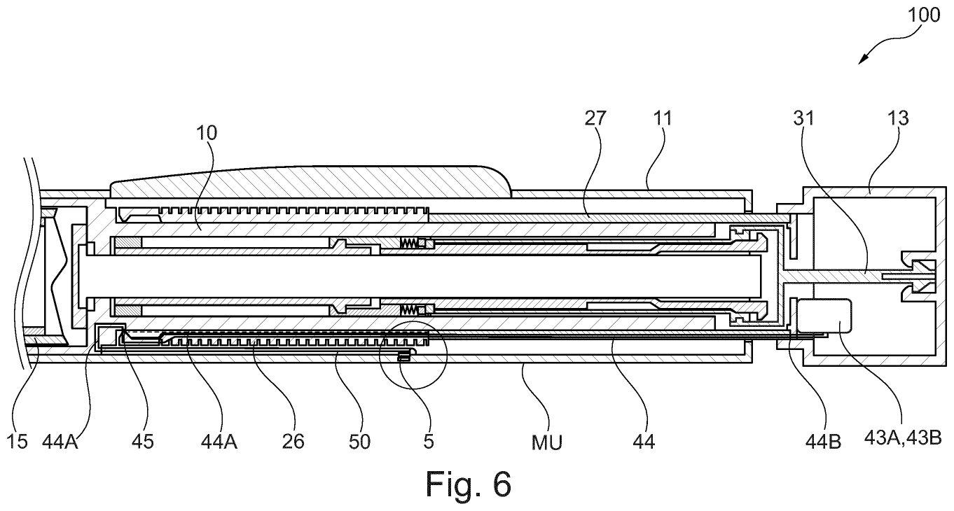

As can be seen in FIG. 2, and in more detail in FIGS. 6 through 9, the mechanism unit MU also includes an electromechanical actuator 5 with an actuator element 50. The actuator element 50 is a displaceable or movable element in the form of a flexible arm. At one longitudinal end, the flexible arm is fixed to the inner body 10 and the other longitudinal end of the arm is a free end which can be displaced in radial direction R. The arm is orientated in an axial direction.

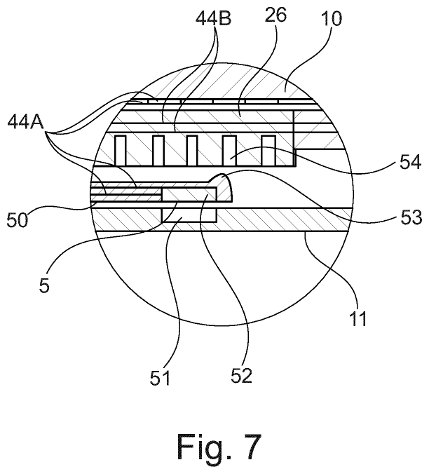

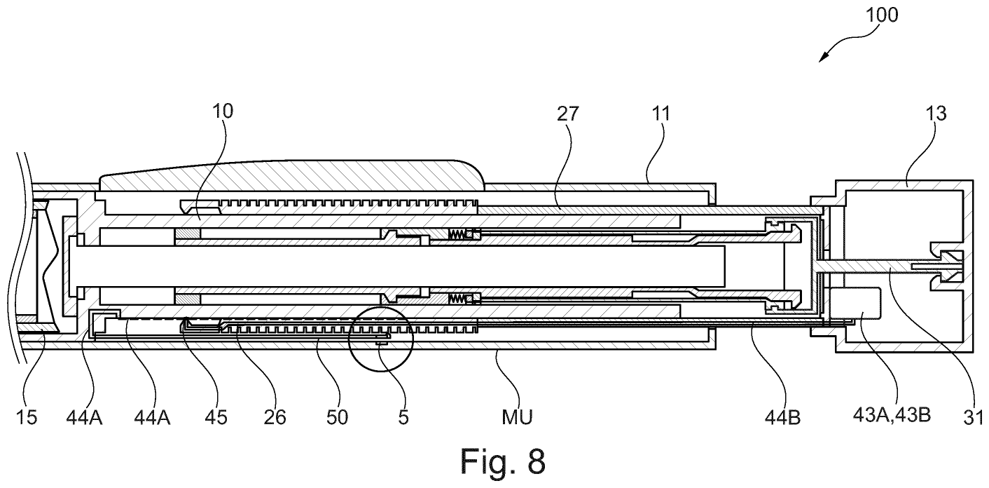

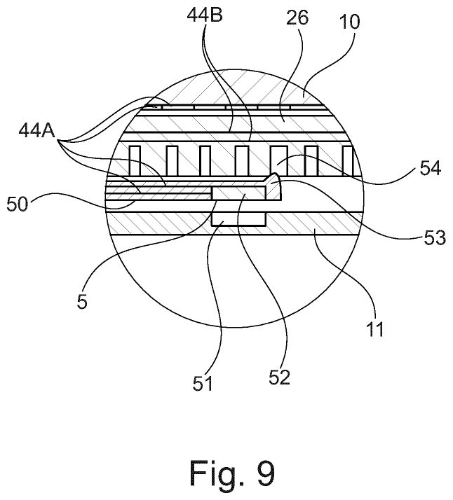

The free longitudinal end of the arm includes an electromagnet 52 (see the detailed view of FIG. 7 illustrating the circled region of FIG. 6 in more detail). The electromagnet 52 is configured to change magnetization when the actuator 5 is operated. The electromagnet 52 is also configured to interact with a magnet 51 in the outer body 11. The magnet 51 axially and/or rotationally overlaps with the electromagnet 52. By changing a current through the electromagnet 52, the magnet's magnetization is changed and the arm can be moved between a first and a second position. FIGS. 6 and 7 show the case where the arm is in the second position (unlocked, first state of the mechanism unit MU). FIGS. 8 and 9 show the case where the arm is in the first position (locked, second state of the mechanism unit MU).

It is indicated in FIGS. 6 through 9 that the number sleeve 26 includes several recesses 54 or grooves which correspond to the amount, set position and pitch of the possible dose units which can be set with the mechanism unit MU (e.g., 24 units). The arm includes a radially inwardly directed protrusion 53. The protrusion 53 is configured to engage into the recesses 54 to block a helical movement between the number sleeve 26 and the arm. As the arm is rotationally and axially fixed to the inner body 10, this engagement will prevent a helical movement of the number sleeve 26 relative to the inner body 10.

As explained with respect to FIG. 1, setting and dispensing a drug dose is associated with a helical movement of the number sleeve 26. Therefore, with the arm in the first position (see FIGS. 8 and 9), the block interface between the arm and the number sleeve 26 prevents setting and dispensing a drug dose. The operational state of the mechanism unit MU is a locked state. When the arm is in the second position (FIGS. 6 and 7), the block interface is released, setting, and dispensing a drug dose is enabled and the operational state of the mechanism unit MU is an unlocked state.

FIGS. 6 and 8 further illustrates how the actuator 5 can be operated. A conductor path 44 is guided from the control unit 43A to the electromagnet 52. By sending an electric current through the conductor path 44 or by changing an electric current in the conductor path 44, the magnetization of the electromagnet 52 may be changed from repelling the magnet 51 to attracting the magnet 51 or vice versa. Controlling an electric current in the conductor path 44 may be done by the control unit 43A. For example, the control unit 43A is configured to operate the actuator 5 by changing the current in the conductor path 44 and to thereby change the operation state of the mechanism unit MU (from the locked state to the unlocked state or vice versa), when a selected drug reservoir unit RU with the contact element 4 at the correct positions is coupled with the mechanism unit MU as explained above.

Instead of using a control unit 43A for operating the actuator 5, the actuator 5 can be automatically operated, for example supplied with electrical current, if the conductor path 41 is closed so that an electric current is transmitted to the electromagnet 52 which changes the magnetization thereof.

As an example, in FIGS. 8 and 9, the electromagnet 52 is not magnetized so that the electromagnet 52 and the magnet 51 do not magnetically interact. The flexible arm is in the first position which may be a relaxed state. When operating the actuator 5, the electromagnet 52 is supplied with current and is then attracted by the magnet 51. The flexible arm moves in radial outward direction into the second position (FIGS. 6 and 7). In this second position, the flexible arm is pre-biased towards the first position. If the operation of the actuator 5 is interrupted by removing the current for the electromagnet 52, the flexible arm automatically returns into the first position.

As can be further seen in FIGS. 6 through 9, the conductor path 44 from the control unit 43A to the electromagnet 52 includes two sections 44A, 44B, which move relative to each other during setting and dispensing a drug dose. The first section 44A is assigned to the inner body and is fixed to the inner body 10. The second section 44B is assigned to the dial sleeve 27 and the number sleeve 26 and moves on a helical path when setting and dispensing a drug dose. To always have an electric contact between the two sections 44A, 44B of the conductor path 44, a sliding contact 45 connects the two sections 44A, 44B. The first section 44A of the conductor path 44 includes a helical conductor track arranged at the inner body 11 which has the same pitch as the helical path on which the dial sleeve 27 and the number sleeve 26 move during setting and dispensing a drug dose.

It can be advantageous to at least partially use the same conductor track, e.g., the same helical conductor track, for the conductor path 41 and the conductor path 44. In this case, the control unit 43A may be configured to differentiate between a current for operating the actuator 5 and a current for verification whether a selected drug reservoir unit RU is coupled to the mechanism unit MU. The differentiation may be based on different frequencies of the different currents. However, we note that systems using just one of the conductor paths 41 and 44 are also within the scope of the present disclosure.

Operation of the actuator 5 may require, additionally or instead of having coupled a selected drug reservoir unit RU, the mechanism unit MU to receive an enabling signal from an external device, like a smartphone or a smartwatch. For this purpose, the mechanism unit MU may include a communication module which is, e.g., arranged on the PCB. The communication module may be a wireless communication module, like a Bluetooth module. If the communication module receives an enabling signal from the external device, the control unit 43A may operate the actuator 5 or may enable operation of the actuator 5. For example, the external device may first be used to read a code, like a QR code, e.g., on the drug reservoir unit RU. The external device may then evaluate, based on the read code, if the drug reservoir unit RU is indeed intended for the user, and may then send the enabling signal to operate the actuator 5.

We note that the conductor paths 41, 44 or sections thereof could also be included by the number sleeve 26, given that the dial sleeve 27 and the number sleeve 26 are expediently axially and rotationally fixed to one another or can be implemented by one unitary component.

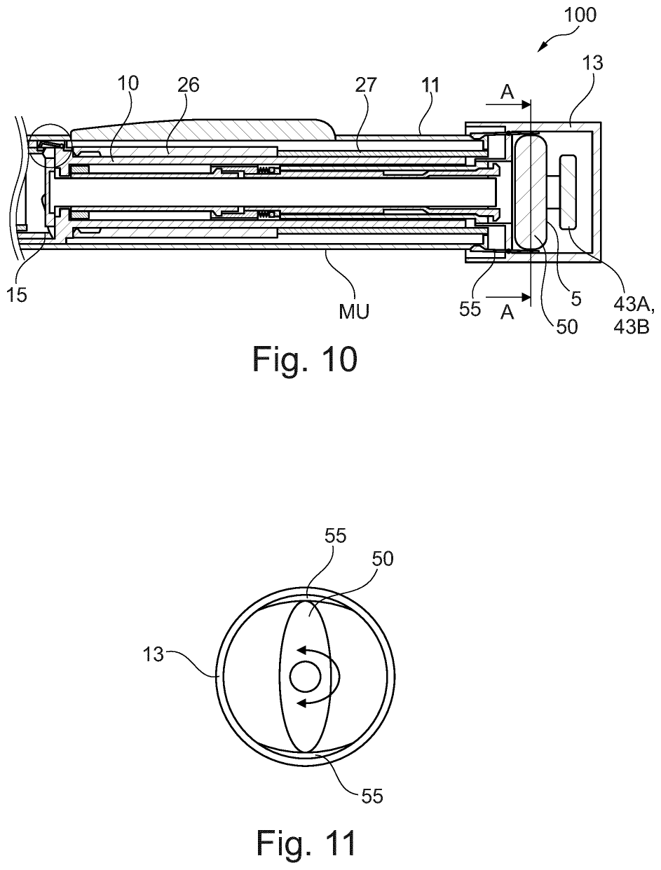

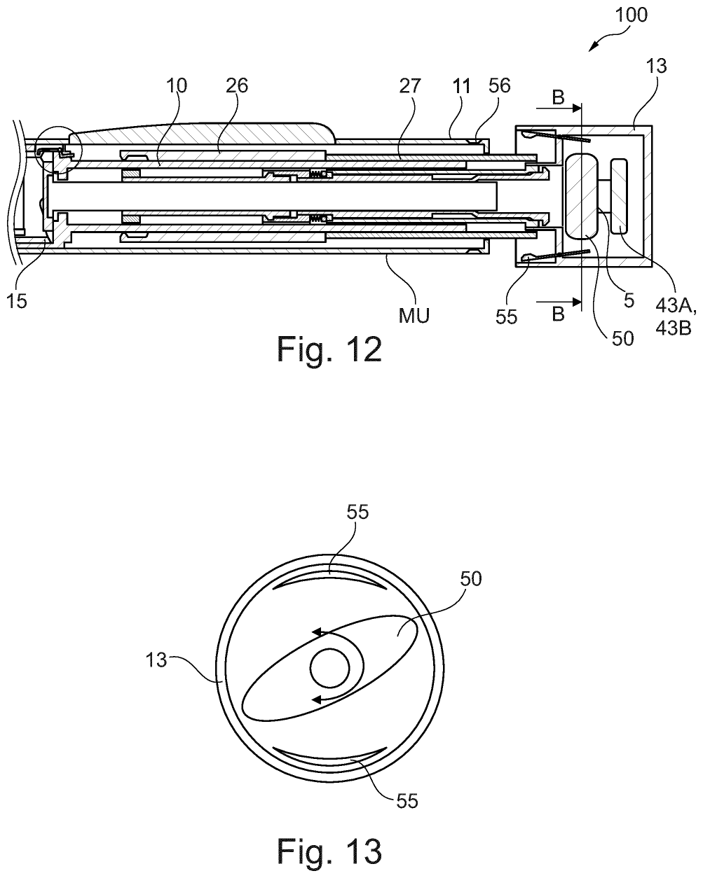

FIGS. 10 through 13 show a second exemplary embodiment of the drug delivery device 100. FIGS. 11 and 13 show cross-sectional views on the planes AA and BB of FIGS. 10 and 12, respectively. The functionalities of this second exemplary embodiment, especially concerning the setting and dispense mechanism, may be similar to the first exemplary embodiment. The actuator 5 for blocking dose setting and/or dose dispensing is, however, different to the one of the first exemplary embodiment.

In the second exemplary embodiment of the drug delivery device 100, the control unit 43A, the battery 43B and also the PCB are coupled and fixed to the knob 13 so that they move together with the knob 13 during setting and dispensing a drug dose. The actuator 5 includes an actuator element 50 in the form of an elliptical disc. This elliptical disc may be rotated with help of an electromotor of the actuator 5. The electromotor is electrically coupled to the control unit 43A so that the control unit 43A can operate the electromotor to rotate the elliptical disc.

FIGS. 10 and 11 show the elliptical disc in a first position. In this first position, the elliptical disc holds an intermediate element in the form of clamps 55 in a respective lock position. The clamps 55 are coupled to the knob 13 such that they are fixed to the knob 13 in axial and rotational direction but are movable with respect to the knob 13 in radial direction. For example, the clamps 55 are pivotably suspended in the knob 13. This is achieved by connecting the clamps 55 via joint connections to the knob 13 so that the clamps 55 can be pivoted relative to the knob 13.

When the elliptical disc is in the first position, the longitudinal ends of the elliptical disc abut against the clamps 55 in radial outward direction which holds the clamps 55 in the lock position. In this lock position, distal ends of the clamps 55 engage into recesses 56 of the outer body 11. This forms a block interface, preventing an axial movement of the knob 13 relative to the outer body 11. As explained in connection with FIG. 1, setting and dispensing a drug dose requires an axial movement of the knob 13 relative to the outer body 11. Thus, the block interface formed between the clamps 55 held in the lock position and the outer body 11 prevents setting and dispensing of a drug dose.

In FIGS. 12 and 13, the actuator 5 has been operated so that the elliptical disc has been rotated from the first position into a second position in which the elliptical disc does no longer hold the clamps 55 in their respective lock position. The mechanism unit MU has thereby changed from a locked state to an unlocked state. This enables the clamps 55 to move from their lock positions into release positions. The movement of the clamps 55 may happen automatically if the clamps 55 are pre-biased towards the release position. With the clamps 55 no longer held in the lock position, the engagement between the distal ends of the clamps 55 and the recesses 56 can be released so that the block interface is released and, accordingly, movement of the knob 13 in the proximal direction P and/or distal direction D for dose setting or dose dispensing, respectively, is enabled.

As for the first exemplary embodiment, operation of the actuator 5 may only be enabled if a selected drug reservoir unit RU, e.g., with contact elements 4 at correct positions, is coupled to the mechanism unit MU so that the conductor path 41 is closed and/or if an enabling signal of an external device is received by the mechanism unit MU.

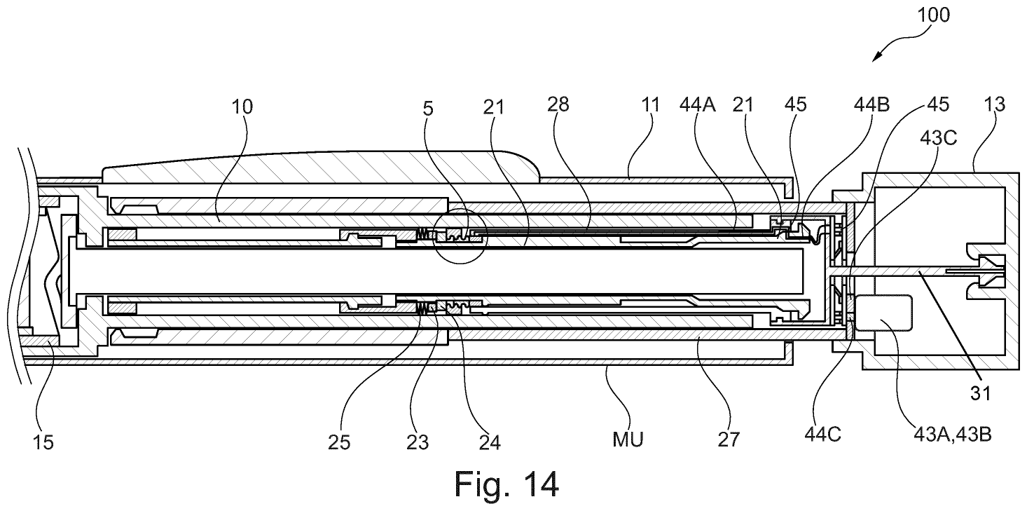

FIGS. 14 through 17 show a third exemplary embodiment of the drug delivery device 100. Also here, the functionalities, especially concerning the setting and dispense mechanism, may be similar to the previously described exemplary embodiments. However, the actuator 5 for blocking and releasing dose setting may be different.

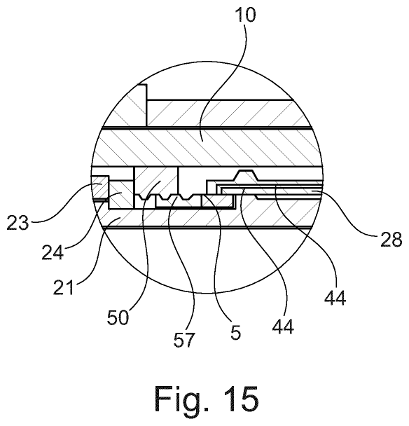

FIG. 14 shows the proximal section of the drug delivery device 100 and FIG. 15 shows the circular region of FIG. 14 in more detail. The actuator 5 in this case includes an actuator element 50 in the form of a spindle nut. The actuator 5 is coupled to the clutch 28. The actuator 5 further includes a spindle 57 which is rotatable by an electromotor of the actuator 5. The spindle 57 and the spindle nut are threadedly engaged so that a rotation of the spindle 57 results in an axial movement of the spindle nut in the distal direction D or proximal direction P, depending on the rotational direction of the spindle 57.

As can be seen in FIGS. 14 and 15, the actuator 5 is electrically connected to the control unit 43A via a conductor track 44 which includes multiple sections, three sections 44A, 44B, 44C in the depicted embodiment. These sections 44A, 44B, 44C are assigned to different elements of the mechanism unit MU. In this case, the first section 44A is assigned to the clutch 28 and the third section 44C is assigned to control unit 43A, the conductor carrier 43C and/or the battery 43B. The section 44C expediently extends in the knob 13. Section 44B may be assigned to the drive sleeve, e.g., the proximal drive sleeve 21 The sections may be connected electrically with one another by contacts 45, e.g., by sliding contacts permitting relative axially and/or rotational movement of the connected components while maintaining a conductive connection between the sections of the conductor track 44 or by non-sliding contacts. Section 44C may be connected to section 44B via a sliding contact 45 which permits relative rotation and, preferably, e.g., on account of the elasticity of conductor elements thereof or separate spring elements, limited relative axial movement, e.g., sufficient to decouple the dial sleeve 27 rotationally from the clutch 28 for dose delivery. Section 44B may include or be connected to one or more wires which bridge a gap to the sliding contact 45. The rotational sliding contact 45 may be arranged between a proximal surface of the clutch coupler 31 and a distal surface of the control unit or the battery or the PCB or conductor carrier 43C. Sections 44A and section 44B are expediently connected by another contact 45, e.g., a sliding contact (such as an axial sliding contact) or a non-sliding contact. Via the contacts and the sections current may be transferred to the actuator 5. The control unit 43A, the battery 43B, and the conductor carrier 43C are arranged in the knob 13. The control unit and the battery may be secured to the dial sleeve 27 in this embodiment, e.g., by fixing the PCB or conductor carrier 43C to the dial sleeve 27. That is to say, the knob 13 is axially and/or rotationally movable relative to the battery and or the control unit in this embodiment and the control unit and/or the battery (along with the PCB) is rotatable relative to the knob. Relative axial movement between the knob 13 and the dial sleeve 27 or the PCB, control unit and/or the battery may be used decouple the clutch rotationally from the drive sleeve, as described herein. Relative rotational movement may occur during the dose delivery operation. To provide the coupling between clutch 28 and knob 13 the clutch coupler extends through the opening in the PCB or conductor carrier 43C. A conductive connection between section 44B of conductor track 44 and the (sliding) contact 45 which may be disposed proximally offset from the drive sleeve, e.g., the proximal drive sleeve 21, may be affected via an opening in the clutch coupler 31, e.g., by wires extending through the opening.

In FIGS. 14 and 15, the spindle nut is in a first position in which the nut presses an intermediate element, namely the proximal clicker 24, in the distal direction D into a lock position. The proximal clicker 24 thereby also presses the distal clicker 23 in the distal direction D and this all happens against the force of the clutch spring 25, thereby compressing the clutch spring 25. As a result of this, the clutch spring 25 presses against the distal clicker 24 in the proximal direction P and in this way the distal clicker 24 and the proximal clicker 24 are pressed against each other. As the faces of the clickers 23, 24 facing each other are toothed, the two clickers 23, 24 pressed against each other cannot be rotated relative to each other. Moreover, since the proximal clicker 24 is splined to the inner body 10, since the distal clicker 23 is splined to the drive sleeve 21 and since the drive sleeve 21 has to be rotated during dose setting, dose setting is prevented with the spindle nut in this fist position.

A distal movement of the proximal clicker 24 relative to the proximal drive sleeve 21, which occurs when the spindle nut is in the first position and/or when the knob 13 is pressed in the distal direction D, may also spline the proximal clicker 24 to the proximal drive sleeve 21 which additionally blocks rotation of the proximal drive sleeve 21 relative to the inner body 10. This may be the case in all exemplary embodiments described herein.

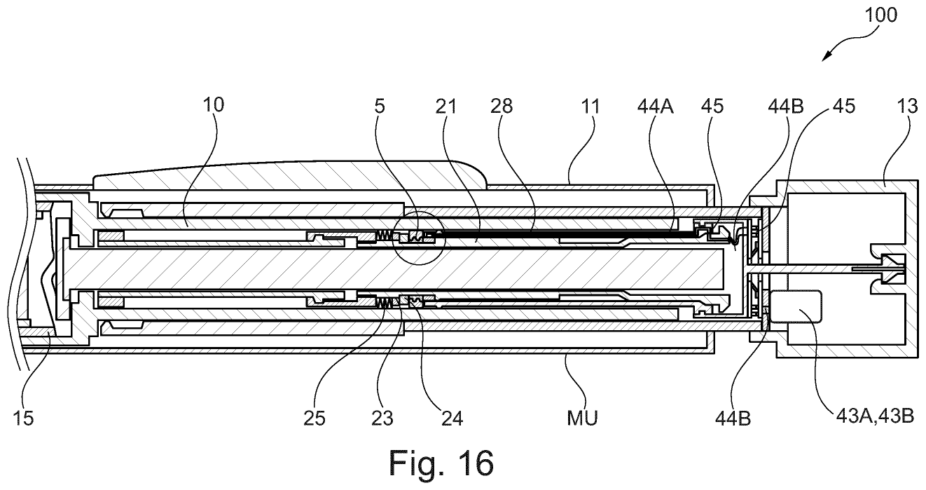

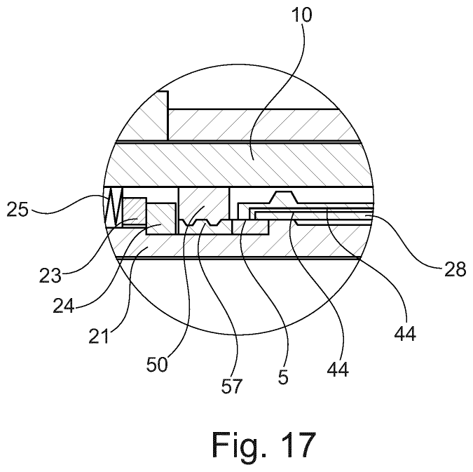

FIGS. 16 and 17 show the spindle nut in a second position after the actuator 5 has been operated so that the spindle nut has been moved in the proximal direction P. The clutch spring 25 is decompressed such that the clickers 23 and 24 are no longer pressed against each other and/or the proximal clicker 24 is no longer splined to the proximal drive sleeve 21.

Consequently, rotation of the proximal drive sleeve 21 and with that dose setting is no longer prevented by the actuator 5.

The operation of the actuator 5 may again be controlled by the control unit 43A. This may again be done dependent on whether a selected drug reservoir unit RU is coupled to the mechanism unit MU and/or dependent on whether an enabling signal of an external device has been received.

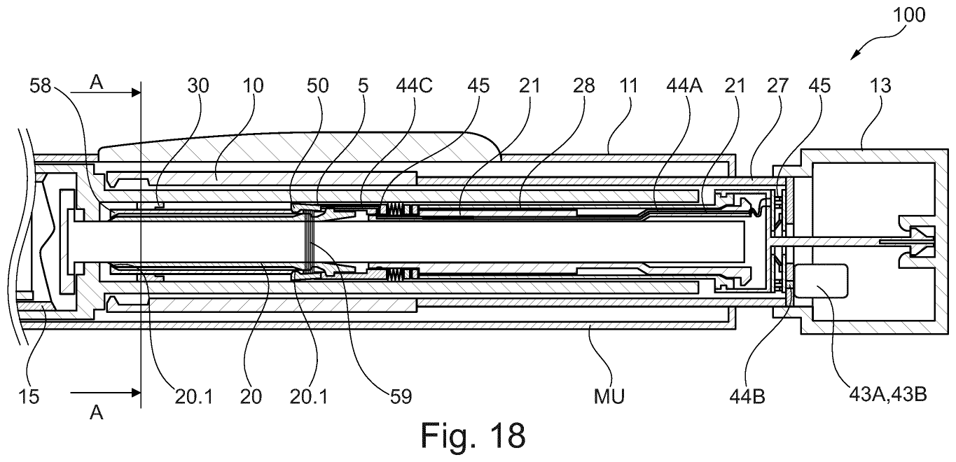

FIGS. 18 through 21 show a fourth exemplary embodiment of the drug delivery device 100. Again, the functionalities, especially concerning the setting and dispense mechanism, may be similar to the previous exemplary embodiments. The actuator 5 for blocking and releasing dose setting is, however, different.

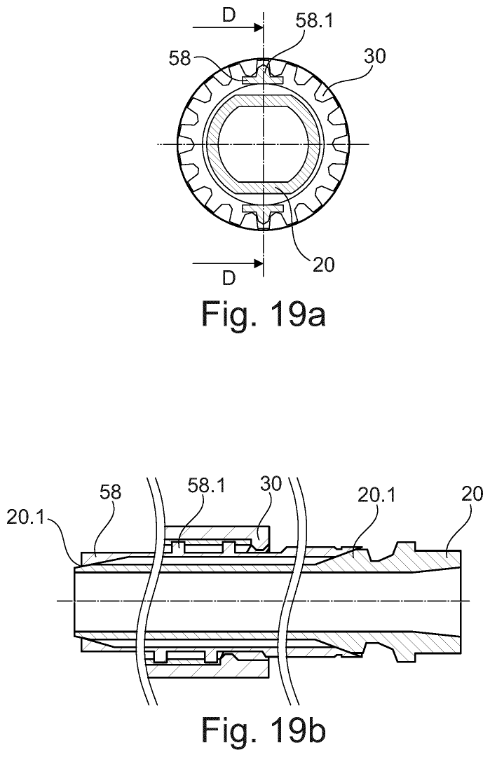

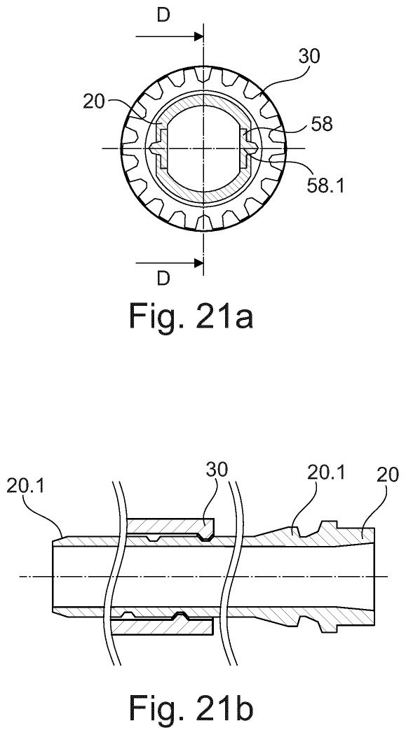

The mechanism unit MU includes an intermediate element in the form of a blocking sleeve 58 which partially surrounds the distal drive sleeve 20. The blocking sleeve 58 includes two elongated arms each with a wedge 58.1 protruding in radial outward direction (see FIGS. 19a and 21a). The distal drive sleeve 20 includes ramps 20.1. The actuator 5 includes actuator elements 50 in the form of actuator arms. The actuator arms can be moved with help of an electromotor of the actuator 5. The actuator 5 is coupled to the drive sleeve coupler 22 and the actuator arms are engaged with the blocking sleeve 58. A blocking sleeve spring 59 biases the blocking sleeve in the distal direction D.

FIG. 19a shows a view on the cross-sectional plane AA of FIG. 18. The inner surface of the last dose nut 30 includes a number of recesses. For example, the number of recesses is equal to or a whole fraction of the number of dose steps in one dose setting revolution. FIG. 19b shows the view on the cross-sectional plane DD of FIG. 19a.

FIGS. 18 and 19 show the drug delivery device 100 with the actuator arms in a first position. The actuator arms may remain in this first position after a short energization of the actuator 5. The actuator arms in the first position have pulled and/or hold the blocking sleeve 58 in a lock position in which the blocking sleeve 58 is pulled over the ramps 20.1 of the distal drive sleeve 20. Thereby, the arms of the blocking sleeve 58 have been forced by the ramps 20.1 to move radially outwardly so that the wedges 58.1 engage into the recesses of the last dose nut 30. Thereby, a block interface is established which blocks relative rotation between the last dose nut 30 and the blocking sleeve 58. The blocking sleeve 58 is rotationally locked to the distal drive sleeve 20. Since the last dose nut 30 cannot rotate relative to the inner body 10, rotation of the distal drive sleeve 20 is prevented with the actuator arms in the first position and the blocking sleeve 58 in the lock position. Thus, setting of a drug dose is prevented. As can be further seen in FIG. 18, with the blocking sleeve 58 in the lock position, the blocking sleeve spring 59 is compressed.

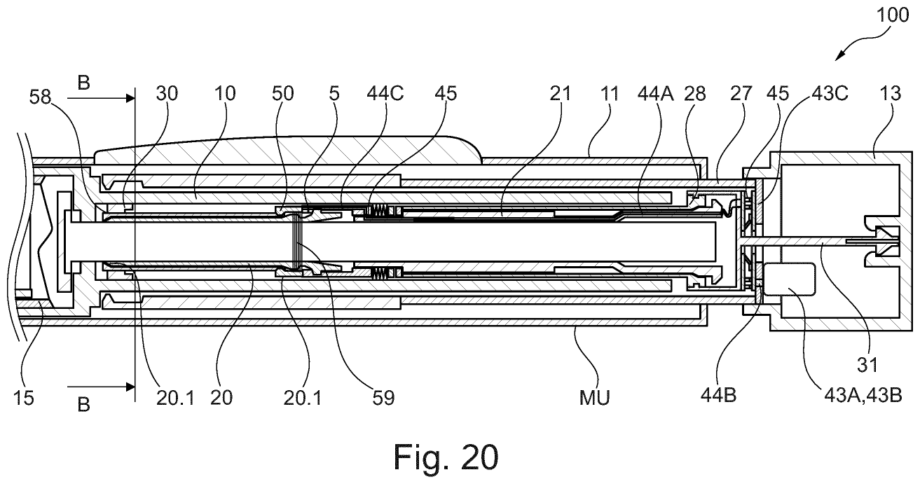

FIGS. 20 and 21 show the drug delivery device 100 with the actuator arms in a second position in which they do no longer hold the blocking sleeve 58 in a lock position. FIG. 21a is the view on the cross-sectional plane BB of FIG. 20. FIG. 21b is the view on the cross-sectional plane CC of FIG. 21a.

The blocking sleeve spring 59 has pushed the blocking sleeve 58 in the distal direction D so that the arms of blocking sleeve 58 are no longer held over the ramps 20.1 and can relax in radial inward direction into a release position. In the release position of the arms of the blocking sleeve 58, the wedges 58.1 of the blocking sleeve 58 do no longer engage into the recesses of the last dose nut 30 so that the block interface is released and rotation of the drive sleeve 20 relative to the last dose nut 30 is allowed. In this way, dose setting is enabled.

FIGS. 18 and 20 also show the electrical connection between the actuator 5 and the control unit 43A or the battery 43B, respectively. The conductor path 44 connecting the actuator 5 with the control unit 43A and/or the battery 43B includes three sections 44A, 44B, 44C which are assigned to different elements of the drug delivery device 100. The control unit 43A, the battery 43B and the section 44B is arranged in the knob 13 and/or connected to the PCB or conductor carrier 43C. The section 44A is fixed to the proximal drive sleeve 21. The section 44C is fixed to the drive sleeve coupler 22. An electrical connection between the sections 44A and 44B and 44A and 44C is maintained during dose dialing and/dose dispensing by contacts 45, e.g., sliding contacts or non-sliding contacts as explained herein in conjunction with FIGS. 14 through 17. The control unit and battery, e.g., via the PCB or conductor carrier 43C, may be fixed to the dial sleeve 27 in this embodiment as has been described above in the context of FIGS. 14 through 17.

The operation of the actuator 5 may again be controlled by the control unit 43A. This may again be done dependent on whether a selected drug reservoir unit RU is coupled with the mechanism unit MU and/or dependent on whether an enabling signal of an external device has been received.

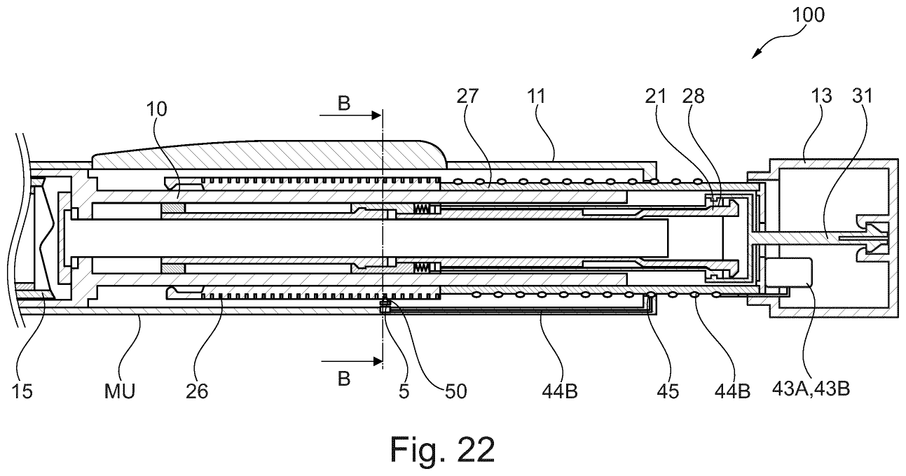

FIGS. 22 through 25 illustrate a fifth exemplary embodiment of the drug delivery device 100. The functionalities, especially concerning the setting and dispense mechanism, may be similar to the previous exemplary embodiments. The actuator 5 is, however, different.

In the fifth exemplary embodiment, the actuator 5 is similar to the actuator 5 of the first exemplary embodiment. Also here, the actuator 5 includes an actuator element 50 in the form of a flexible arm which is at one longitudinal end fixed to the outer body 11 and has one free longitudinal end. A magnet 51 is arranged at the free longitudinal end of the arm. An electromagnet 52 is coupled to the outer body 11 and configured to interact with the magnet 51 of the arm.

One difference from the actuator 5 of the first exemplary embodiment is that the arm according to the fifth exemplary embodiment is orientated circumferentially instead of axially. For example, the arm extends over at least 90° or at least 150°. A further difference is that the electromagnet 52 is not arranged at the arm but fixed to the outer body 11. However, an arrangement with the electromagnet 52 coupled to the arm and the magnet 51 assigned to the outer body 11 is also conceivable.

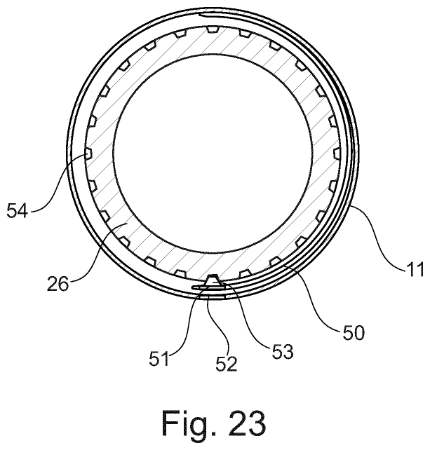

FIGS. 22 and 23 illustrate the drug delivery device 100 with the arm in a first position. FIG. 23 is a view on the cross-sectional plane BB of FIG. 22. In the first position, a radially inwardly directed protrusion 53 of the arm engages into a recess 54 of the number sleeve 26. The recesses 54 in the number sleeve 26 correspond to the amount and pitch of the possible dose units which can be set with the mechanism unit MU. The arm may be in a relaxed state and/or may be held in the first position due to a repellent interaction between the magnets 51, 52. With the arm in the first position engaging with the number sleeve 26, relative rotation between the number sleeve 26 and the outer body 11 is blocked so that dose setting and/or dose dispensing is prevented.

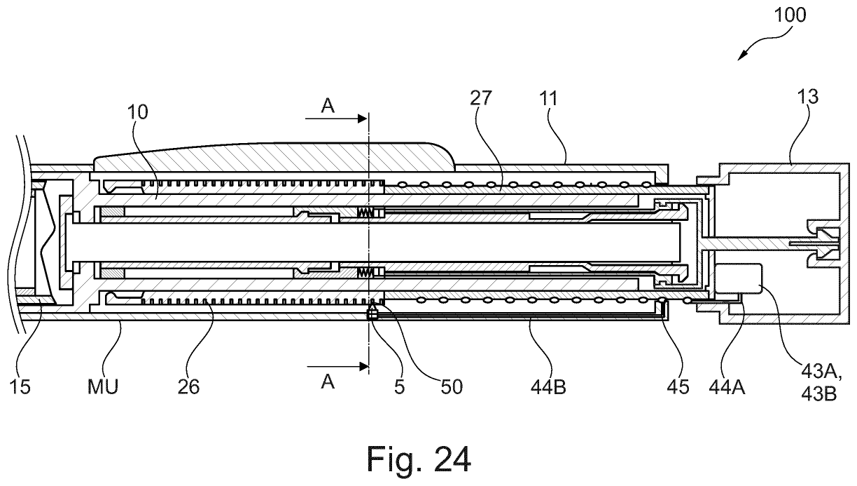

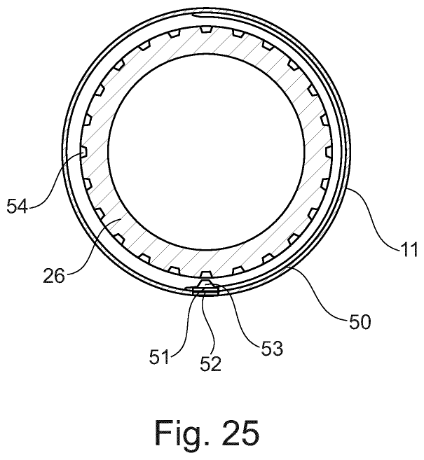

FIGS. 24 and 25 show the drug delivery device 100 of FIGS. 22 and 23 in the same views as FIGS. 22 and 23 but now with the arm in a second position. The arm may move into this second position when operating the actuator 5 so that the magnetization of the electromagnet 52 is changed. For example, the magnetization of the electromagnet 52 is changed such that the two magnets 51 and 52 now attract each other so that the arm is moved in radial outward direction by the attractive force between the magnets 51, 52. Thereby, the engagement between the protrusion 53 and the recess 54 is released and the number sleeve 26 is no longer prevented from rotation relative to the outer body 11. Thus, dose setting and/or dose dispensing is enabled.

FIGS. 22 and 24 also illustrates the design of the conductor path 44 from the control unit 43A and/or battery 43B to the electromagnet 52. Like in the first exemplary embodiment, the control unit 43A, the battery 43B and the PCB are fixed to the dial sleeve 27. The conductor path 44 includes two sections 44A and 44B which are movable relative to each other during dose setting and dose dispensing and are electrically connected via a sliding contact 45. The first section 44A of the conductor path 44 includes a helical conductor track with the same pitch as the helical path on which the dial sleeve 27 and the number sleeve 26 move during dose setting and/or dose dispensing.

The operation of the actuator 5 may again be controlled by the control unit 43A. This may again be done dependent on whether a selected drug reservoir unit RU is coupled with the mechanism unit MU and/or dependent on whether an enabling signal of an external device has been received.

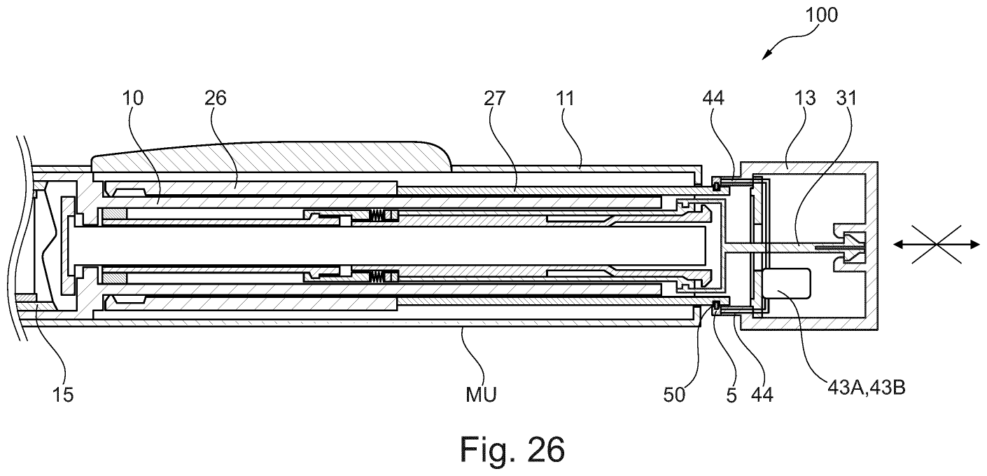

FIGS. 26 through 28 show a sixth exemplary embodiment of the drug delivery device 100. Again, this exemplary embodiment may have similar functionalities, especially concerning the setting and dispense mechanism, as the previous exemplary embodiments, but deviates from the previous exemplary embodiments in the design of the actuator 5.

In the sixth exemplary embodiment, the control unit 43A and/or the battery 43B are coupled to the knob 13 so that they move together with the knob 13. The actuator 5 is also part of the knob 13. The actuator element 50 of the actuator 5 is, e.g., a pin which can be moved in radial direction by the actuator 5.

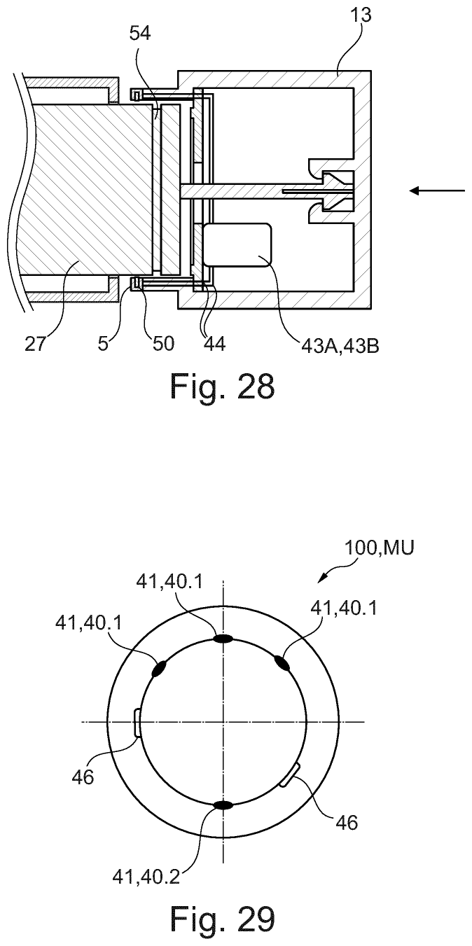

In FIG. 26, the pin is in a first position in which the pin engages into a recess 54, particularly a ring groove of the dial sleeve 27 (see also FIG. 28). Due to this engagement, relative axial movement between the knob 13 and the dial sleeve 27 is prevented. Consequently, dose dispensing is prevented, because the splined interface between the clutch 28 and the dial sleeve 27 cannot be released.

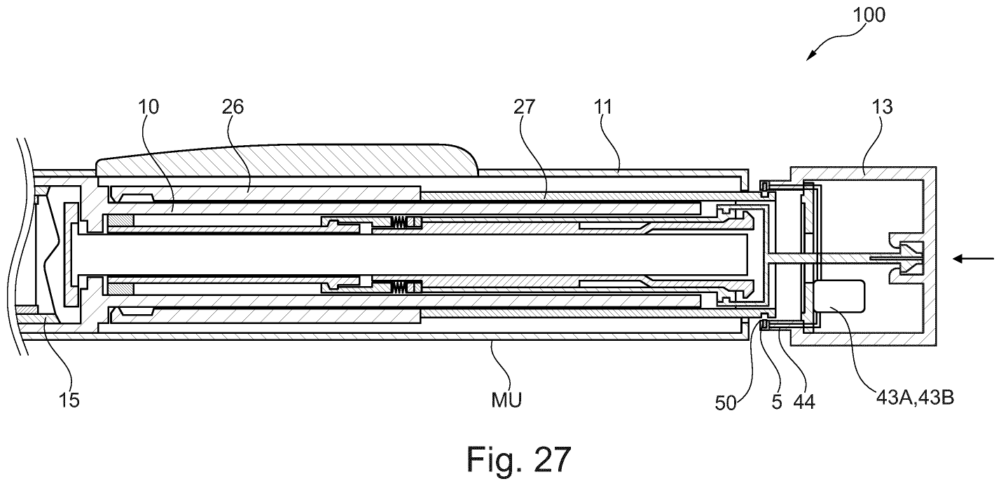

FIG. 27 shows the drug delivery device 100 with the pin in a second position in which the pin does not longer engage into the recess 54. Relative axial and rotational movement between the knob 13 and the dial sleeve 27 are allowed and therefore dose dispensing is enabled.

In FIG. 28, the configuration with the knob 13 pushed in distal direction is shown. The knob 13 has slightly moved relative to the dial sleeve 27 in the distal direction D which is needed for drug dispensing as these releases the splined interface between the clutch 28 and the dial sleeve 27.

Also here, the operation of the actuator 5 may be controlled by the control unit 43A. This may again be done dependent on whether a selected drug reservoir unit RU is coupled with the mechanism unit MU and/or dependent on whether an enabling signal of an external device has been received.

Some or all of the actuators 5 described in connection with the first to the sixth exemplary embodiment may also be combined.

FIG. 29 shows an exemplary embodiment of the drug delivery device 100 or of the mechanism unit MU, respectively. Shown is a cross-sectional view on a plane running perpendicularly to the longitudinal axis. FIG. 29 may show any one of the first to sixth exemplary embodiment.

The mechanism unit MU is configured to be coupled with three different kinds of selected drug reservoir units RU, to prevent operation of the actuator 5 unless a selected drug reservoir unit RU is coupled thereto and/or to enable operation of the actuator 5 in case any one of the three selected drug reservoir units RU is coupled thereto. For this purpose, the mechanism unit includes three different conductor paths 41, each having a first contact point 40.1 and a second contact point 40.2. The second contact point 40.2 (lower one in FIG. 29) and the associated conductor path section is the same for all three conductor paths 41. The first contact points 40.1 of the different conductor paths 41 (upper ones in FIG. 29) and the associated conductor path sections are different. Particularly, the first contact points 40.1 of the different conductor paths 41 are offset with respect to each other in rotational direction but do overlap in radial and axial direction.

If a selected drug reservoir unit RU with a contact element 4, particularly with access points 4.1, 4.2, at a correct position (see FIG. 30) is coupled with the mechanism unit MU, one of the three conductor paths 41 is closed and this can be recognized, e.g., by the control unit 43A of the mechanism unit MU as described in connection with the first exemplary embodiment. The control unit 43A may then operate an actuator 5 of the mechanism unit MU to change an operational state of the mechanism unit MU, e.g., from a locked state, in which dose setting and/or dose dispensing is prevented, to an unlocked state, in which dose setting and/or dose dispensing is enabled.

If a drug reservoir unit with no contact element 4 at a correct position is coupled with the mechanism unit MU, a change of the operational state may not be prevented.

The structure of the mechanism unit MU including several conductor paths 41 and associated contact points 40 for different kind of selected drug reservoir units RU can be realized by any of the previously described exemplary embodiments.

FIG. 30 shows cross-sectional views of exemplary embodiments of the three different kinds of selected drug reservoir units RU for the mechanism unit MU of FIG. 29. Each of the three selected drug reservoir units RU has a contact element 4 at a different position, particularly with access points 4.1, 4.2 of the contact elements 4 at different positions. A second access point 4.2 of each of the contact elements 4 is always at the same position but first access points 4.1 of the contact elements 4 are at different positions, particularly at different angular positions. The access points 4.1, 4.2 of a drug reservoir unit RU may be electrically connected via the contact element 4.

The positions of the access points 4.1, 4.2 of a selected drug reservoir unit RU match with the positions of the contact points 40.1, 40.2 of one conductor path 41 so that when a selected drug reservoir unit RU is coupled with the mechanism unit MU this conductor path 41 is closed via the contact element 4 of the drug reservoir unit RU.

To make sure that, when coupling a selected drug reservoir unit RU with the mechanism unit MU, the orientation of the drug reservoir unit RU, particularly in rotational direction, is correct, the mechanism unit MU includes guiding structures 46 in the form of guide grooves (see FIG. 29) which are configured to engage with guide structures 47 in the form of guide ribs of the drug reservoir units RU. This ensures that the access points 4.1, 4.2 always contact the associated contact points 40.1, 40.2 when a selected drug reservoir unit RU is coupled with the mechanism unit MU.

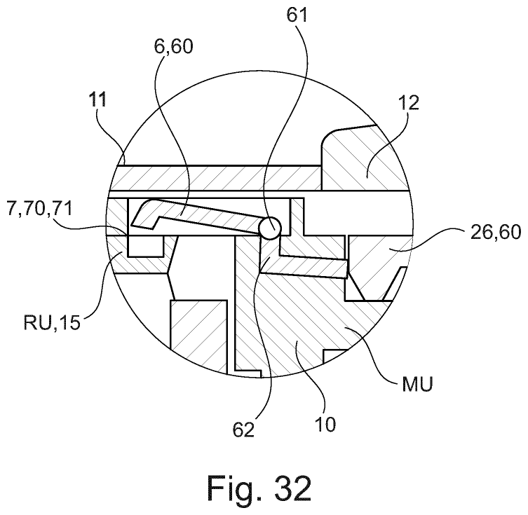

FIGS. 31 and 32 show the circled regions of FIGS. 12 and 10, respectively, in greater detail. These figures indicate the functional principle of a locking mechanism 6 configured to prevent disconnecting or separating the drug reservoir unit RU from the mechanism unit MU when a drug dose is set but not fully dispensed.

The inner body 10 includes an interface feature, e.g., in the form of an inner thread 70. The drug reservoir unit RU, in this case the reservoir holder 15 of the drug reservoir unit RU, includes an interface feature, e.g., in the form of an outer thread 71. The two threads 70, 71 may be engaged and thereby establish a connection interface 7 in the form of a threaded interface via which the drug reservoir unit RU is releasably connected to the mechanism unit MU. For releasing the connection and the connection interface 7, the drug reservoir unit RU may have to be rotated and/or moved in the proximal direction P or distal direction D with respect to the body 10, 11.

In FIG. 31, however, the release of the connection interface 7 is prevented by a locking mechanism 6 being in a locked state. The locking mechanism 6 includes a coupling element 60 which is in a lock position in which the coupling element 60 engages with the reservoir holder 15. The coupling element 60 includes a coupling feature in the form of a protrusion at the distal end of the coupling element 60, which engages into a coupling feature, namely a recess or groove, of the reservoir holder 15. This engagement prevents that the drug reservoir unit RU can be moved axially, optionally also rotationally, with respect to the body 10, 11 so that release of the connection interface 7 is prevented.

The coupling element 60 is pivotably suspended in the mechanism unit MU via a joint connection 61 to the inner body 10. Due to this joint connection 61, the coupling element 60 can be rotated out of the lock position of FIG. 31 into a release position shown in FIG. 32.

The coupling element 60 is an elongated element with a main section running in an axial direction and a further section 62 running perpendicularly to the main section and to the rotational axis around which the coupling element 60 is rotatable. The coupling element 60 is arranged such that the number sleeve 26, which constitutes a part of the locking mechanism 6 and which moves in an axial direction during dose setting and dose dispensing, can, when the number sleeve 26 reaches a first position (see FIG. 32) hit the coupling element 60 radially offset of the joint connection 61 to exert a torque onto the coupling element 60. Due to this torque, the coupling element 60 is moved out of the lock position into the release position of FIG. 32. This happens mechanically via a leverage effect.

As can be seen in FIG. 32, when the number sleeve 26 is in the first position and the coupling element 60 accordingly is in the release position, the coupling element 60 is no longer engaged with the drug reservoir unit RU and release of the connection interface 7 is enabled. This state of the locking mechanism 6 is called released state. A user can now separate the drug reservoir unit RU form the mechanism unit MU, e.g., to exchange the drug reservoir unit RU. When the number sleeve 26 is moved in the proximal direction P, e.g., during dose setting, the coupling element 60 automatically returns into a lock position and release of the connection interface 7 would again be prevented.

The locking mechanism 6 described in connection with FIGS. 31 and 32 is particularly useful for preventing a user to change the drug reservoir unit RU when a drug dose is set. As mentioned, setting a drug dose is associated with a movement of the number sleeve 26 in the proximal direction P. This locking mechanism 6 may be used in any of the exemplary embodiments of a drug delivery device described herein.

The concepts proposed in the present disclosure, e.g., the ones for blocking setting and/or dispensing of a dose or the remaining concepts, may not only be applied to the device architecture described above, e.g., in the most detail in conjunction with FIG. 1 and the associated embodiment, but also to other drug delivery devices. In particular one or more of the presently proposed concepts could be applied to devices disclosed in WO 2021/059202 A1, e.g., to implement the function described in [00237] thereof, or devices disclosed in EP 3 049 132 B1, see claim 1.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders.

Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g., a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des (B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g., a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody,” as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., the antibody has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full-length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

The disclosure described herein is not limited by the description in conjunction with the exemplary embodiments. Rather, the disclosure includes any new feature as well as any combination of features, particularly including any combination of features in the patent claims, even if said feature or said combination per se is not explicitly stated in the patent claims or exemplary embodiments.

### REFERENCE NUMERALS

4 contact element
4.1 first access point
4.2 second access point
5 actuator
6 locking mechanism
7 connection interface
10 inner body
11 outer body
12 window
13 dose button
14 cap
15 cartridge holder
16 cartridge container
17 stopper
20 distal drive sleeve
20.1 ramp
21 proximal drive sleeve
22 drive sleeve coupler
23 distal clicker
24 proximal clicker
25 clutch spring
26 number sleeve
27 dial sleeve
28 clutch
29 plunger rod
30 last dose nut
31 clutch coupler
40 contact point
40.1 first contact point
40.2 second contact point
41 conductor path
41A first section of conductor path 41
41B second section of conductor path 41
42 sliding contact
43A control unit
43B battery
43C conductor carrier/PCB
44 conductor path
44A first section of conductor path 44
44B second section of conductor path 44
44C third section of conductor path 44
45 sliding contact
46 guiding structure
47 guide structure
50 actuator element
51 magnet
52 magnet
53 protrusion
54 recess
55 clamp
56 recess
57 spindle
58 blocking sleeve
58.1 wedge
59 blocking sleeve spring
60 coupling element
61 joint connection
62 section of coupling element 60
70 interface feature
71 interface feature
100 drug delivery device
MU mechanism unit
RU drug reservoir unit
D distal direction
P proximal direction
L longitudinal axis
R radial direction
C azimuthal direction/rotational direction/angular direction

1-21. (canceled)

22. A drug delivery device, comprising:
a mechanism unit with:
a housing element,
a first movable element arranged movable with respect to the housing element, and
an electromechanical actuator that, when operated, moves an actuator element between a first position and a second position, wherein the mechanism unit is configured:
to be operatively coupled with a drug reservoir unit and to enable a dispense process for dispensing a drug dose,
to enable setting a drug dose to be dispensed, wherein setting a drug dose is associated with a movement of the first movable element in a first direction,
to block movement of the first movable element in the first direction when the actuator element is in the first position in order to prevent setting a drug dose, and
to allow movement of the first movable element in the first direction when the actuator element is in the second position as a precondition for setting a drug dose.

23. The drug delivery device according to claim 22, wherein the mechanism unit is configured to prevent dispensing a drug dose when the actuator element is in the first position.

24. The drug delivery device according to claim 23, wherein:
dispensing a drug dose is associated with a movement of the first movable element in a second direction, and
the mechanism unit is configured to block movement of the first movable element in the second direction when the actuator element is in the first position.

25. The drug delivery device according to claim 22, wherein:
the mechanism unit is configured such that, unless a selected drug reservoir unit is coupled with the mechanism unit, operation of the actuator is prevented, and a coupling of the mechanism unit with a selected drug reservoir unit is a precondition for an operation of the actuator, and/or
the mechanism unit comprises a first conductor path, the first conductor path comprises at least one contact point for electrically contacting at least one contact element of a drug reservoir unit such that when a selected drug reservoir unit with a contact element at a correct position is coupled with the mechanism unit, the at least one contact point electrically contacts the contact element which changes an electrical property of the first conductor path, and the mechanism unit is configured such that operation of the actuator is prevented unless the electrical property of the first conductor path is changed.

26. The drug delivery device according to claim 25, wherein:
when a selected drug reservoir unit is coupled with the mechanism unit, the first conductor path is closed, and
the closed first conductor path electrically connects at least one of:
the actuator with a control unit for the actuator,
the control unit with an energy source,
the actuator with the energy source, or
an output interface of the control unit with an input interface of the control unit.

27. The drug delivery device according to claim 25, wherein:
the first conductor path comprises at least two sections which are (i) movable with respect to each other, and (ii) electrically connected by a sliding contact.

28. The drug delivery device according to claim 22, wherein:
the mechanism unit further comprises a communication module for communicating with an external device, and
the mechanism unit is configured such that operation of the actuator is prevented unless an enabling signal from the external device is received via the communication module.

29. The drug delivery device according to claim 22, wherein:
the first movable element is rotated and/or moved axially when being moved in the first direction during setting a drug dose.

30. The drug delivery device according to claim 22, wherein:
the mechanism unit further comprises an intermediate element displaceable between a lock position and a release position, and
movement of the actuator element from the first position into the second position enables a movement of the intermediate element from the lock position into the release position and/or vice versa,
the intermediate element in the lock position is configured to block movement of the first moveable element in the first direction, and/or
a path along which the actuator element moves between the first and the second position is different from a path along which the intermediate element moves between the lock position and the release position.

31. The drug delivery device according to claim 22, wherein:
the drug delivery device comprises a dose setting member configured to be operated by a user in order to set a drug dose, and
during dose setting, the dose setting member is moved relative to the housing element.

32. The drug delivery device according to claim 22, wherein:
an energy source and/or a control unit for operating the actuator and/or the actuator element are moved during setting a drug dose.

33. The drug delivery device according to claim 22, wherein:
the mechanism unit further comprises a second conductor path for guiding an electrical signal to the actuator, and
the second conductor path comprises at least two sections which are (i) movable with respect to each other, and (ii) electrically connected by a sliding contact.

34. The drug delivery device according to claim 22, wherein:
the actuator comprises a magnet and an electromagnet a magnetization of which is changed when the actuator is operated,
magnetic interaction between the magnet and the electromagnet results in movement of the actuator element between the first position and the second position when the actuator is operated and/or magnetic interaction holds the actuator element in the first position.

35. The drug delivery device according to claim 22, wherein:
the actuator element is a displaceable or movable element in form of a flexible arm, and
the flexible arm comprises an electromagnet at a free longitudinal end thereof, and/or
the flexible arm is orientated in axial direction or circumferential direction.

36. The drug delivery device according to claim 22, wherein:
the actuator comprises an actuator element in form of an elliptical disc or a spindle, and
the actuator element is a spindle nut which is moved by the spindle.

37. The drug delivery device according to claim 22, wherein the mechanism unit comprises a first conductor path which is interrupted, unless a selected drug reservoir unit is coupled with the mechanism unit.

38. The drug delivery device according to claim 22, wherein the drug delivery device is an injection device such as an autoinjector and/or a variable dose device or a fixed dose device and/or a pen type device, in particular a dial extension pen.

39. The drug delivery device according to claim 22, wherein:
the drug delivery device comprises the drug reservoir unit, and
the drug reservoir unit (i) comprises a drug container with a drug, or (ii) is configured to retain a drug container with a drug.

40. The drug delivery device according to claim 22, wherein:
the mechanism unit comprises a dispense mechanism for dispensing a drug dose,
the dispense mechanism comprises a plunger rod and an energy member,
the energy member is configured to provide energy for moving the plunger rod in distal direction, and/or
the energy member is a drive spring, a gas cartridge or an electric motor.

41. A method for dispensing a drug with a drug delivery device, wherein the drug delivery device comprises:
a drug reservoir unit, wherein the drug reservoir unit (i) comprises a drug container with the drug, or (ii) is configured to retain a drug container with the drug, and
a mechanism unit with:
a housing element,
a first movable element arranged movable with respect to the housing element,
an electromechanical actuator that, when operated, moves an actuator element between a first position and a second position, wherein the mechanism unit is configured:
to be operatively coupled with the drug reservoir unit and to enable a dispense process for dispensing a drug dose,
to enable setting a drug dose to be dispensed, wherein setting a drug dose is associated with a movement of the first movable element in a first direction,
to block movement of the first movable element in the first direction when the actuator element is in the first position in order to prevent setting a drug dose, and
to allow movement of the first movable element in the first direction when the actuator element is in the second position as a precondition for setting a drug dose.
